(19) United States
(12) Patent Application Publication
(10) Pub. No.: US 2025/0206838 A1
(43) Pub. Date: Jun. 26, 2025

(54) THERAPEUTIC INHIBITION OF CD38 TO TREAT HIDRADENITIS SUPPURATIVA

(71) Applicant: HENRY FORD HEALTH SYSTEM, Detroit, MI (US)

(72) Inventors: Qing-Sheng MI, Bloomfield Hills, MI (US); Li ZHOU, Bloomfield Hills, MI (US); Peter DIMITRION, Detroit, MI (US); Iltefat HAMZAVI, Northville, MI (US)

(21) Appl. No.: 18/848,184

(22) PCT Filed: Mar. 17, 2023
(86) PCT No.: PCT/US2023/064652

§ 371 Date: Sep. 18, 2024

Related U.S. Application Data
(60) Provisional application No. 63/321,416, filed on Mar. 18, 2022.

(51) Int. Cl. C07K 16/28; A61K 45/06

(52) U.S. Cl. CPC C07K 16/2896; A61K 45/06; C07K 2317/76

(57) ABSTRACT

Methods of preventing and treating Hidradenitis suppurativa include administering an antagonist of CD38 to a subject likely of developing Hidradenitis suppurativa or a subject having one or more symptoms associated with Hidradenitis suppurativa, or a subject diagnosed by a medical professional as having Hidradenitis suppurativa.

### CROSS REFERENCE TO RELATED APPLICATIONS

This PCT International Application claims the benefit of, and priority to U.S. Provisional Application No. 63/321,416, filed on Mar. 18, 2022, the disclosure of which is incorporated by reference herein in its entirety.

### TECHNICAL FIELD

The present disclosure, in some embodiments, relates to a method of treatment of Hidradenitis Suppurativa (HS) by parenteral or topical administration to a subject in need thereof, a composition comprising an anti-CD38 antagonist as an active agent. The compositions of this invention are useful for the treatment, prevention or amelioration of HS, also known as acne inversa.

### BACKGROUND

Hidradenitis suppurativa (HS) is a chronic inflammatory skin disease that can have a devastating impact on an individual's quality of life. Patients with HS have exceptionally high pain scores and often develop disfiguring lesions that can lead to social embarrassment and even depression. The prevalence of HS is estimated to be about 1%, and the condition trends with ethnic background and family history, pointing to a key role for inheritance. Genetic studies have identified several disease-causing and disease-susceptibility loci, the most well-studied being those of the 7-secretase complex; however, no definitive genotype-phenotype connections have been established.

Follicular occlusion has been implicated as an inciting event in the pathogenesis of HS, but shifting paradigms and recent studies point toward HS as an autoinflammatory keratinization disease. In this model, subclinical inflammation promotes keratinization and subsequent occlusion of hair follicles, rather than the other way around.

The number of different cell types that transcriptomic and proteomic studies have implicated in the pathogenesis of HS has grown substantially, suggesting marked heterogeneity and discrete disease subclusters among HS patients. Recent proteomic and transcriptomic studies have shown that circulating inflammatory proteins are upregulated during HS and that the proteomes in the skin and blood show good correlation. These studies highlight features of systemic inflammation in patients with HS.

For studying complex inflammatory diseases with multiple axes of inflammation and multi-factorial etiology such as HS, bulk analyses are fruitful but insufficient to systematically derive cell-type specific changes that may underlie pathogenesis. To date, few researchers have used single-cell technologies to assess individual cell population changes in the skin during HS; however, no single-cell studies have been conducted on the peripheral immune system.

HS is a long-term chronic skin disease whose present treatment options are often unsatisfactory. HS has a profound effect on patient's quality of life (QoL). Alavi A. et al., reviewed QoL aspects of this disease in an article titled “Quality-of-Life Impairment in Patients with Hidradenitis Suppurativa” (Am. J. Clin. Dermatol., 2014, vol. 16. No. 1, pages 61-65). The clinical picture of HS includes solitary nodules, diffuse, painful abscesses, malodorous drainage, sinus tract formation and scarring. The exact cause of HS is still unclear, but it is believed that the underlying mechanism involves dysfunction of the apocrine sweat glands and/or hair follicles.

Current treatment for HS consists of topical and/or systemic antibiotics, hormonal interventions, analgesics and, in selected cases, immunosuppressants, the tumor necrosis factor (TNF-α) inhibitor monoclonal antibody adalimumab, and surgical excision. (Gulliver et al. (2016); Zouboulis et al. (2015) J Eur. Acad. Dermatol. Venereol. 29:619-4414-16; Kimball et al. (2016) N Engl. J Med 375:422-34). However, symptom control and lesion resolution are inconsistent among treatments. The recurrence rate is high after discontinuation of antibiotic therapy and long-term treatment with retinoids poses teratogenicity concerns. Moreover, the effectiveness of inflammatory drugs, such as dapsone, fumarates and cyclosporine, is based on small case studies with varying results. As a result of these inconsistent outcomes, and the severity of the HS disease, HS patients utilize healthcare in high-cost settings (e.g., emergency department and inpatient care) more frequently than patients with other chronic inflammatory skin conditions. (Khalsa et al. (2016) J Am Acad Dermatol 73:609-14; Kirby et al. (2014) JAMA Dermatol 150:937-44). Because there is no medical cure for HS, and the disease is physically and psychologically debilitating, there is a clear unmet need to provide safe and effective long-term treatments for HS patients.

The U.S. Food and Drug Administration (FDA) approved the first treatment for HS in 2015. It is a biologic called adalimumab, a fully human IgG1 monoclonal antibody that specifically binds to tumor necrosis factor (TNF)-alpha, and is administered by subcutaneous injection. In studies, the patients who received adalimumab had noticeably fewer abscesses (lumps with pus) and nodules (hard, deep lumps). The FDA has approved adalimumab for people 12 years of age or older who have moderate to severe HS. However, many HS patients do not respond to adalimumab. There remains a substantial unmet medical need for effective therapies for treating HS.

Daratumumab® (anti-CD38 antibody) and SARCLISA® (isatuximab-irfc, anti-CD38 antibody), have been recently FDA approved for the treatment of relapsed refractory multiple myeloma (RRMM) along with after showing extraordinary efficacy in clinical trials.

### SUMMARY

The present disclosure describes parenteral and topical compositions comprising an anti-CD38 antagonist, and a carrier suitable for parenteral or topical administration for the treatment, prevention and amelioration of symptoms associated with HS. The anti-CD38 antagonist, for example, an anti-CD38 antibody, or an antigen-binding fragment thereof, in the composition of this disclosure is in liquid, or semi-solid form, according to need, and may be administered parenterally or applied topically to the site of HS. In the present disclosure, the inventors have utilized mass cytometry by time-of-flight (CyTOF) to systematically characterize peripheral immune cell changes in the blood of HS patients. Studies have found previously undescribed immune cell dysregulation and a potential role for CD38 in the pathogenesis of HS.

In some embodiments, the composition of this invention is useful for the treatment, prevention or alleviation of Hidradenitis suppurativa (HS).

In some embodiments, the anti-CD38 antagonist (e.g., CD38 antibody or antigen-binding fragment thereof, is subcutaneously (SC) administered at a dose of about 10 mg-about 5,000 mg, e.g., about 10 mg, 25 mg, 50 mg, 75 mg, 100 mg, 150 mg, 200 mg, 250 mg, 300 mg, 350 mg, 400 mg, 380 mg, 500 mg, 550 mg, 600 mg, 650 mg, 700 mg, 750 mg, 800 mg, 850 mg, 900 mg, 1,000 mg, 1,200 mg, 1,400 mg, 1,600 mg, 1,800 mg, 2,000 mg, 2,200 mg, 2,400 mg, 2,600 mg, 2,800 mg, 3,000 mg (e.g., from about 10 mg to about 5,000 mg, and all intervals and integers therebetween) once per day, once per week, once every two weeks, once every four weeks, 2 or more times per week, 2 or more times every 2 weeks, 2 or more times every month, and variations thereof. In some embodiments, the CD38 antagonist (e.g., CD38 antibody or antigen-binding fragment thereof, such as Daratumumab®®) is administered subcutaneously using an induction regimen, followed by a maintenance regimen. In some embodiments, the induction regimen comprises weekly administration and the maintenance regimen comprises administration every two weeks or every four weeks. In some embodiments, the CD38 antagonist (e.g., CD38 antibody or antigen-binding fragment thereof, such as Daratumumab®®) is administered SC at a dose of about 10 mg to about 500 mg, e.g., from about 10 mg to about 300 mg, at week 0, 1, 2, 3, and 4 and then every two weeks thereafter. In some embodiments, the CD38 antagonist (e.g., CD38 antibody or antigen-binding fragment thereof) is administered subcutaneously, (SC) at a dose of about 20 mg to about 200 mg, e.g., 100 mg, at week 0, 1, 2, 3, and 4 and then every four weeks thereafter. In some embodiments, the CD38 antagonist (e.g., CD38 antibody or antigen-binding fragment thereof) is administered SC at a dose of about 10 mg to about 5,000 mg (e.g., 50-2,000 mg) at week 0, 1, 2, 3, and 4 and then every four weeks (monthly) thereafter. In various embodiments, the anti-CD38 antibody, or antigen-binding fragment thereof described in the various methods and dose regimen described above is Daratumumab®.

### BRIEF DESCRIPTION OF THE DRAWINGS

FIG. 1A shows a schematic of the CyTOF immune profiling experimental design.

FIG. 1B shows representative t-SNE-clustering of live singlets highlighted by major immune cell populations identified by bivariate gating.

FIG. 1C shows cell lineage marker expression (CD66b, CD45, CD19, CD3, CD4, CD8, CD11c, CD56, and CD161) on t-SNE plots in HS and HC peripheral blood.

FIG. 1D shows representative t-SNE-clustering of CD45+ cells highlighted by cell type subset identified by bivariate gating.

FIG. 1E shows heatmap of Z-score normalized frequencies with hierarchical clustering. Colored dots below subset name corresponds to the tSNE in FIG. 1D.

FIG. 1F shows principle Components Analysis (PCA) plot of CD45+ subsets shaded by HS and HC.

FIG. 2A1 shows the frequency of total NK cells as a percent of CD45+ cells.

FIG. 2A2 shows the frequency early NK cells as a percent of CD45+ cells.

FIG. 2A3 shows the frequency of late NK cells as a percent of CD45+ cells.

FIG. 2B1 shows the frequency of total CD4 αβ T cells as a percent of CD45+ cells.

FIG. 2B2 shows the frequency of Th1 cells as a percent of CD45+ cells.

FIG. 2B3 shows the frequency of Th2 cells as a percent of CD45+ cells.

FIG. 2B4 shows the frequency of Th17 cells as a percent of CD45+ cells.

FIG. 2C1 shows representative bivariate flow cytometry plots of stimulated PBMCs (4 hours PMA/Iono) from healthy control (HC) and HS pre-gated on live singlet CD3+CD4+ cells to show the expression of IL-17.

FIG. 2C2 shows a graphical representation of the flow cytometry plots of FIG. 2C1.

FIG. 2D1 shows the frequency of total DCs as a percent of CD45+ cells.

FIG. 2D2 shows the frequency of mDCs as a percent of CD45+ cells.

FIG. 2D3 shows the frequency of pDCs as a percent of CD45+ cells.

FIG. 2E1 shows frequency of total monocytes as a percent of CD45+ cells. P values are the result of an unpaired student's t-test.

FIG. 2E2 shows the frequency of classical monocytes as a percent of CD45+ cells. P values are the result of an unpaired student's t-test.

FIG. 2E3 shows the frequency of intermediate monocytes as a percent of CD45+ cells. P values are the result of an unpaired student's t-test.

FIG. 2E4 shows the frequency of nonclassical monocytes as a percent of CD45+ cells. P values are the result of an unpaired student's t-test.

FIG. 3A shows CITRUS clusters from L1-penalized regression LASSO analysis, statistically significant clusters have darker shading.

FIG. 3B1 shows CD3 protein expression plots for lineage markers overlayed on CITRUS clusters.

FIG. 3B2 shows CD19 protein expression plots for lineage markers overlayed on CITRUS clusters.

FIG. 3B3 shows CD4 protein expression plots for lineage markers overlayed on CITRUS clusters.

FIG. 3B4 shows CD8 protein expression plots for lineage markers overlayed on CITRUS clusters.

FIG. 3B5 shows CD11c protein expression plots for lineage markers overlayed on CITRUS clusters.

FIG. 3B6 shows CD14 protein expression plots for lineage markers overlayed on CITRUS clusters.

FIG. 3B7 shows CD16 protein expression plots for lineage markers overlayed on CITRUS clusters.

FIG. 3B8 shows CD56 protein expression plots for lineage markers overlayed on CITRUS clusters.

FIG. 3C shows cells from significant CITRUS clusters mapped to t-SNE plots.

FIG. 3D shows marker expression histograms for cell clusters 188915, 188810, 188628, & 189395. Curves represent cluster expression level and blue curves are the expression of all other CD45+ cells. Annotations were made based on both marker expression and t-SNE localization.

FIG. 3E shows boxplots representing the abundances of each cell type cluster from HS and HC blood.

FIG. 4A shows median expression of CCR6 in immune cell types. Asterisks represent p<0.05 from unpaired student's t tests between HC and HS.

FIG. 4B shows median expression of CCR4 in immune cell types. Asterisks represent p<0.05 from unpaired student's t tests between HC and HS.

FIG. 4C shows median expression of CD38 in immune cell types. Asterisks represent p<0.05 from unpaired student's t tests between HC and HS.

FIG. 5A shows the gating strategy to identify CD38+ monocyte subsets.

FIG. 5B shows the frequency of CD38+ monocytes as a percent of their respective subset P-values reported are a result of unpaired student's t-tests.

FIG. 5C1 shows CCR4 expression stratified by HS vs. HC and CD38hi vs. CD38lo in classical monocytes. P values reported are a result of paired student's t-tests.

FIG. 5C2 shows CCR4 expression stratified by HS vs. HC and CD38hi vs. CD38lo in intermediate monocytes. P values reported are a result of paired student's t-tests.

FIG. 5C3 shows CCR4 expression stratified by HS vs. HC and CD38hi vs. CD38lo in nonclassical monocytes. P values reported are a result of paired student's t-tests.

FIG. 5C4 shows CCR6 expression stratified by HS vs. HC and CD38hi vs. CD38lo in classical monocytes. P values reported are a result of paired student's t-tests.

FIG. 5C5 shows CCR6 expression stratified by HS vs. HC and CD38hi vs. CD38lo in intermediate monocytes. P values reported are a result of paired student's t-tests.

FIG. 5C6 shows CCR6 expression stratified by HS vs. HC and CD38hi vs. CD38lo in nonclassical monocytes. P values reported are a result of paired student's t-tests.

FIG. 5D1 shows frequency of CD38+ classical monocyte subsets as a percent of their respective monocyte subset stratified by Hurley Stage. P values reported are a result of one-way ANOVA with multiple comparisons.

FIG. 5D2 shows frequency of CD38+ intermediate monocyte subsets as a percent of their respective monocyte subset stratified by Hurley Stage. P values reported are a result of one-way ANOVA with multiple comparisons.

FIG. 5D3 shows frequency of CD38+ nonclassical monocyte subsets as a percent of their respective monocyte subset stratified by Hurley Stage. P values reported are a result of one-way ANOVA with multiple comparisons.

FIG. 6A shows counts per million (CPM) of CD38 from HS lesional (L), HS perilesional (PL), HS nonlesional (NL), and healthy control (HC) skin.

FIG. 6B shows heatmap of normalized counts of genes significantly correlated with CD38 expression (Bonferroni corrected FDR<0.05). Samples were ordered by CD38 expression from left to right.

FIG. 6C shows pathways ordered by CD38 expression from left to right. The significantly correlated genes were enriched using MSigDB.

FIG. 6D shows cell types ordered by CD38 expression from left to right. The significantly correlated genes were enriched using the Human Gene Atlas.

FIG. 6E shows functional activity ordered by CD38 expression from left to right. The significantly correlated genes were enriched using gene ontology:molecular function (GO:MF).

FIG. 6F1 shows immunohistochemical staining for CD38 in a lesional HS tissue section. Staining intensity was scored on a scale of 0 to 3 relative to a positive control and negative control.

FIG. 6F2 shows immunohistochemical staining for CD38 in a perilesional HS tissue section. Staining intensity was scored on a scale of 0 to 3 relative to a positive control and negative control.

FIG. 6F3 shows immunohistochemical staining for CD14 in a lesional HS tissue section. Staining intensity was scored on a scale of 0 to 3 relative to a positive control and negative control.

FIG. 6F4 shows immunohistochemical staining for CD14 in a perilesional HS tissue section. Staining intensity was scored on a scale of 0 to 3 relative to a positive control and negative control.

FIG. 6F5 shows immunohistochemical staining for CD56 in a lesional HS tissue section. Staining intensity was scored on a scale of 0 to 3 relative to a positive control and negative control.

FIG. 6F6 shows immunohistochemical staining for CD56 in a perilesional HS tissue section. Staining intensity was scored on a scale of 0 to 3 relative to a positive control and negative control.

FIG. 6F7 shows immunohistochemical staining for CD16 in a lesional HS tissue section. Staining intensity was scored on a scale of 0 to 3 relative to a positive control and negative control.

FIG. 6F8 shows immunohistochemical staining for CD16 in a perilesional HS tissue section. Staining intensity was scored on a scale of 0 to 3 relative to a positive control and negative control.

FIG. 6F9 shows a graphical representation of immunohistochemical staining of CD38 in lesional and perilesional HS and HC tissue. Each point is the average of three scores from independent raters. Two-way student's t test ****p<0.0001; ***p<0.001.

FIG. 6F10 shows a graphical representation of immunohistochemical staining of CD14 in lesional and perilesional HS and HC tissue. Each point is the average of three scores from independent raters. Two-way student's t test ****p<0.0001; ***p<0.001.

FIG. 6F11 shows a graphical representation of immunohistochemical staining of CD56 in lesional and perilesional HS and HC tissue. Each point is the average of three scores from independent raters. Two-way student's t test ****p<0.0001; ***p<0.001.

FIG. 6F12 shows a graphical representation of immunohistochemical staining of CD16 in lesional (L) and perilesional (PL) HS and HC tissue. Each point is the average of three scores from independent raters. Two-way student's t test ****p<0.0001; ***p<0.001.

FIG. 7A shows a heatmap of marker expression in annotated cell clusters, with the bar graph on the right showing the number of cells in each cluster over all captured regions of interest (ROI).

FIG. 7B shows normalized frequency of each cell type in lesional (L) and perilesional (PL) skin: L-Dermis, PL-dermis, L-epidermis, PL-epidermis, and tunnel regions of interest (ROIs).

FIG. 7C shows representative image showing co-localization of CD31, CD14, and CD38 in PL-Dermis and L-Dermis ROIs.

FIG. 7D shows median expression of CD38 from CD38+ monocyte-derived macrophages.

FIG. 7E shows graphical depiction of the patch detection method.

FIG. 7F shows Voronoi images of endothelial cell (EC) patches shaded by unique identification number. The graphical plot shows the number of EC of patches per ROI between lesional (L) and perilesional (PL) ROIs. P value reported is the result of an unpaired student's t test.

FIG. 7G shows Voronoi images of monocyte and monocyte-derived macrophage patches shaded by unique identification number. The graphical plot shows the number of monocyte and monocyte-derived macrophage (mono/mono-mac) of patches per ROI between lesional (L) and perilesional (PL) ROIs. P-value reported is the result of an unpaired student's t test.

FIG. 7H shows the percent of overlapping endothelial cells (EC) and monocyte and monocyte-derived macrophage (mono/mono-mac) patches in lesional (L) and perilesional (PL) ROIs. P value reported is the result of an unpaired student's t test.

FIG. 7I shows the cellular composition of endothelial cell (EC) patches. Top: frequency of cells per patch. Bottom: Spearmann correlation of immune cell frequencies in endothelial cell (EC) patches. Left: lesional-endothelial cell patches (L-EC patches). Right: perilesional-endothelial cell patches (PL-EC patches).

FIG. 7J shows median scaled expression of functional markers on immune cell subsets found in lesional (L) (left) and perilesional (PL) (right) endothelial cell (EC) patches.

FIG. 8A shows a schematic of the CyTOF immune profiling workflow to identify immune profile predictors of anti-TNFα responders and non-responders.

FIG. 8B shows the frequency of CD38+ intermediate monocytes (I.Mos) out of the total intermediate monocytes is elevated in patients that fail to respond to anti-TNF α.

FIG. 8C shows the ROC analysis of CD38+ intermediate monocyte (I.Mo) frequency between responders (R) and non-responders (NR) to anti-TNFα; comparison highlights that CD38+ intermediate monocytes (I.Mos) are a positive predictor of patients who are anti-TNFα non-responders.

FIG. 8D shows the frequency of IgD+ memory B cells (Mem. B) between anti-TNFα responders (R) and non-responders (NR), (*p<0.05).

### DETAILED DESCRIPTION

Throughout this specification, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or group of compositions of matter.

Disclosed herein are novel anti-CD38 antibodies, antigen binding fragments thereof, and conjugates thereof (e.g., antibody drug conjugates; ADCs) that are useful, e.g., because they cross-react between human CD38 and non-human primate CD38. Further, anti-CD38 antibodies, and fragments thereof, described herein can be used as therapeutic agents. For example, anti-CD38 antibodies, fragments thereof, and anti-CD38 ADCs can be used to treat patients with conditions for which depletion of CD38+ cells is beneficial, including, but not limited to, leukemias and lymphomas, as well as patients with autoimmune diseases such as multiple sclerosis and scleroderma. In addition, the anti-hematopoietic cell antibodies (anti-CD38 antibodies) included hereincri are useful in hematopoietic stem cell therapies. For example, the antibodies or ADCs herein are useful in conditioning procedures, in which a patient is prepared for receipt of a transplant including hematopoietic stem cells. Such procedures promote the engraftment of a hematopoietic stem cell transplant. According to the methods described herein, a patient may be conditioned for hematopoietic stem cell transplant therapy by administration to the patient of an anti-CD38 ADC, antibody or antigen-binding fragment thereof capable of binding CD38 (e.g., CD38 expressed by hematopoietic cells (e.g., hematopoietic stem cells or mature immune cells (e.g., B cells, T cells, monocytes, NK cells). As described herein, the anti-CD38 antibody may be covalently conjugated to a cytotoxin so as to form an antibody drug conjugate (ADC). Administration of an ADC capable of binding CD38 to a patient in need of treatment, for example, by selectively depleting endogenous CD38+ cells, for example, CD38⁺ monocytes, B cells, NK cells, and/or endothelial cells.

The sections that follow provide methods of treatment of HS, by administering a therapeutic regimen comprising anti-CD38 antagonists, for example, anti-CD38 antibodies, and fragments thereof. In some embodiments, the anti-CD38 antagonists, have cross reactivity with both human and non-human primate CD38. The sections that follow also provide a description of the anti-CD38 antibodies, or antibody conjugates thereof, that can be administered to a patient, such as a patient suffering from HS and/or symptoms of HS, as well as methods of administering such anti-CD38 antagonist therapeutics to a patient (e.g., a HS patient).

### Definitions

As used herein, the term “about” refers to a value that is within 5% above or below the value being described. For example, the term “about 100 nM” indicates a range of 95-105 nM.

As used herein, the term “allogeneic”, in the context of transplantation, is used to define a transplant (e.g., cells, tissue or an organ transplant) that is transplanted from a donor to a recipient, wherein the recipient is a different individual of the same species, relative to the donor.

As used herein, the term “autologous”, in the context of transplantation, refers to a transplant where the donor and recipient are the same individual, i.e., the same subject.

As used herein, the term “xenogeneic”, in the context of transplantation, refers to a transplant where the donor and recipient are of different species.

As used herein, the term “immune cell” is intended to include, but is not limited to, a cell that is of hematopoietic origin and that plays a role in the immune response. Immune cells include, but are not limited to, T cells and natural killer (NK) cells. Natural killer cells are well known in the art. In one embodiment, natural killer cells include cell lines, such as NK-92 cells. Further examples of NK cell lines include NKG, YT, NK-YS, HANK-1, YTS cells, and NKL cells. An immune cell can be allogeneic or autologous.

As used herein, the term “antibody” refers to an immunoglobulin molecule that specifically binds to, or is immunologically reactive with, a particular antigen. An antibody includes, but is not limited to, monoclonal antibodies, polyclonal antibodies, multispecific antibodies (e.g., bispecific antibodies), and antibody fragments so long as they exhibit the desired antigen-binding activity.

Generally, antibodies comprise heavy and light chains containing antigen binding regions. Each heavy chain is comprised of a heavy chain variable region (abbreviated herein as HCVR or VH) and a heavy chain constant region. The heavy chain constant region is comprised of three domains, CH1. CH2 and CH3. Each light chain is comprised of a light chain variable region (abbreviated herein as LCVR or VL) and a light chain constant region. The light chain constant region is comprised of one domain, CL. The VH, and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

The term “antigen-binding fragment,” or “antigen binding portion” of an antibody, as used herein, refers to one or more portions of an antibody that retain the ability to specifically bind to a target antigen. The antigen-binding function of an antibody can be performed by fragments of a full-length antibody. The antibody fragments can be, for example, a Fab, F(ab′)2, scFv, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the VL, VH, CL, and CH1 domains; (ii) a F(ab′)2 fragment, a bivalent fragment containing two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb including VH and VL domains; (vi) a dAb fragment that consists of a VH domain (see, e.g., Ward et al., Nature 341:544-546, 1989); (vii) a dAb which consists of a VH or a VL domain; (viii) an isolated complementarity determining region (CDR); and (ix) a combination of two or more (e.g., two, three, four, five, or six) isolated CDRs which may optionally be joined by a synthetic linker. Furthermore, although the two domains of the Fv fragment, VL and VH, are coded for by separate genes, they can be joined, using recombinant methods, by a linker that enables them to be made as a single protein chain in which the VL and VH regions pair to form monovalent molecules (known as single chain Fv (scFv); see, for example, Bird et al., Science 242:423-426, 1988 and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art.

An “intact” or “full length” antibody, as used herein, refers to an antibody having two heavy (H) chain polypeptides and two light (L) chain polypeptides interconnected by disulfide bonds. In certain embodiments, a toxin can be conjugated to an intact anti-CD38 antibody having heavy and/or light chain amino acid sequences described herein.

The term “monoclonal antibody” as used herein refers to an antibody that is derived from a single clone, including any eukaryotic, prokaryotic, or phage clone, by any means available or known in the art, and is not limited to antibodies produced through hybridoma technology. Monoclonal antibodies useful with the present disclosure can be prepared using a wide variety of techniques known in the art including the use of hybridoma, recombinant, and phage display technologies, or a combination thereof.

The terms “Fc region,” “Fc domain,” and “IgG Fc domain” as used herein refer to the portion of an immunoglobulin, e.g., an IgG molecule, that correlates to a crystallizable fragment obtained by papain digestion of an IgG molecule. The Fc region comprises the C-terminal half of two heavy chains of an IgG molecule that are linked by disulfide bonds. It has no antigen binding activity but contains the carbohydrate moiety and binding sites for complement and Fc receptors, including the FcRn receptor (see below). For example, an Fc domain contains the entire second constant domain CH2 (residues at EU positions 231-340 of IgG1) and the third constant domain CH3 (residues at EU positions 341-447 of human IgG1). As used herein, the Fc domain includes the “lower hinge region” (residues at EU positions 233-239 of IgG1).

Fc can refer to this region in isolation, or this region in the context of an antibody, antibody fragment, or Fc fusion protein. Polymorphisms have been observed at a number of positions in Fc domains, including but not limited to EU positions 270, 272, 312, 315, 356, and 358, and thus slight differences between the sequences presented in the instant application and sequences known in the art can exist. Thus, a “wild type IgG Fc domain” or “WT IgG Fc domain” refers to any naturally occurring IgG Fc region (i.e., any allele). The sequences of the heavy chains of human IgG1, IgG2, IgG3 and IgG4 can be found in a number of sequence databases, for example, at the Uniprot database (www.uniprot.org) under accession numbers P01857 (IGHG1_HUMAN), P01859 (IGHG2_HUMAN), P01860 (IGHG3_HUMAN), and P01861 (IGHG1_HUMAN), respectively.

The terms “modified Fc region” or “variant Fc region” as used herein refers to an IgG Fc domain comprising one or more amino acid substitutions, deletions, insertions or modifications introduced at any position within the Fc domain. In certain aspects a variant IgG Fc domain comprises one or more amino acid substitutions resulting in decreased or ablated binding affinity for an Fc gamma R and/or C1q as compared to the wild type Fc domain not comprising the one or more amino acid substitutions. Further, Fc binding interactions are essential for a variety of effector functions and downstream signaling events including, but not limited to, antibody dependent cell-mediated cytotoxicity (ADCC) and complement dependent cytotoxicity (CDC). Accordingly, in certain aspects, an antibody comprising a variant Fc domain (e.g., an antibody, fusion protein or conjugate) can exhibit altered binding affinity for at least one or more Fc ligands (e.g., Fc gamma Rs) relative to a corresponding antibody otherwise having the same amino acid sequence but not comprising the one or more amino acid substitution, deletion, insertion or modifications such as, for example, an unmodified Fc region containing naturally occurring amino acid residues at the corresponding position in the Fc region.

Variant Fc domains are defined according to the amino acid modifications that compose them. For all amino acid substitutions discussed herein in regard to the Fc region. numbering is always according to the EU index as in Kabat Thus, for example, D265C is an Fc variant with the aspartic acid (D) at EU position 265 substituted with cysteine (C) relative to the parent Fc domain. It is noted that the order in which substitutions are provided is arbitrary.

The terms “Fc gamma receptor” or “Fc gamma R” as used herein refer to any member of the family of proteins that bind the IgG antibody Fc region and are encoded by the FcgammaR genes. In humans this family includes but is not limited to FcgammaRI (CD64), including isoforms FcgammaRIa, FcgammaRIb, and FcgammaRIc; FcgammaRII (CD32), including isoforms FcgammaRIIa (including allotypes H131 and R131), FcgammaRIIb (including FcgammaRIIb-1 and FcgammaRIIb-2), and FcgammaRIIc; and FcgammaRIII (CD16), including isoforms FcgammaRIIIa (including allotypes V158 and F158) and FcgammaRIIIb (including allotypes FcgammaRIIIb-NA1 and FcgammaRIIIb-NA2), as well as any undiscovered human FcgammaRs or FcgammaR isoforms or allotypes. An FcgammaR can be from any organism, including but not limited to humans, mice, rats, rabbits, and monkeys. Mouse FcgammaRs include but are not limited to FcgammaRI (CD64), FcgammaRII (CD32), FcgammaRIII (CD16), and FcgammaRIII-2 (CD16-2), as well as any undiscovered mouse FcgammaRs or FcgammaR isoforms or allotypes.

The term “effector function” as used herein refers to a biochemical event that results from the interaction of an Fc domain with an Fc receptor. Effector functions include but are not limited to ADCC, ADCP, and CDC. By “effector cell” as used herein is meant a cell of the immune system that expresses or one or more Fc receptors and mediates one or more effector functions. Effector cells include but are not limited to monocytes, macrophages, neutrophils, dendritic cells, eosinophils, mast cells, platelets, B cells, large granular lymphocytes, Langerhans' cells, natural killer (NK) cells, and gamma-delta T cells, and can be from any organism included but not limited to humans, mice, rats, rabbits, and monkeys.

The term “silent”, “silenced”, or “silencing” as used herein refers to an antibody having a modified Fc region described herein that has decreased binding to an Fc gamma receptor (FcγR) relative to binding of an identical antibody comprising an unmodified Fc region to the FcγR (e.g., a decrease in binding to a FcγR by at least 70%, at least 80%, at least 90%, at least 95%, at least 98%, at least 99%, or 100% relative to binding of the identical antibody comprising an unmodified Fc region to the FcγR as measured by, e.g., BLI). In some embodiments, the Fc silenced antibody has no detectable binding to an FcγR. Binding of an antibody having a modified Fc region to an FcγR can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE® analysis or Octet™ analysis (forteBIO)), and other methods such as indirect binding assays, competitive binding assays fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions. One example of a competitive binding assay is a radioimmunoassay comprising the incubation of labeled antigen with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound in the presence of increasing amounts of an unlabeled second antibody.

As used herein, the term “identical antibody comprising an unmodified Fc region” refers to an antibody that lacks the recited amino acid substitutions (e.g., D265C, H435A), but otherwise has the same amino acid sequence as the Fc modified antibody to which it is being compared.

The terms “antibody-dependent cell-mediated cytotoxicity” or “ADCC” refer to a form of cytotoxicity in which a polypeptide comprising an Fc domain, e.g., an antibody, bound onto Fc receptors (FcRs) present on certain cytotoxic cells (e.g., primarily NK cells, neutrophils, and macrophages) and enables these cytotoxic effector cells to bind specifically to an antigen-bearing “target cell” and subsequently kill the target cell with cytotoxins. (Hogarth et al., Nature review Drug Discovery 2012, 11:313) It is contemplated that, in addition to antibodies and fragments thereof, other polypeptides comprising Fc domains, e.g., Fc fusion proteins and Fc conjugate proteins, having the capacity to bind specifically to an antigen-bearing target cell will be able to effect cell-mediated cytotoxicity.

For simplicity, the cell-mediated cytotoxicity resulting from the activity of a polypeptide comprising an Fc domain is also referred to herein as ADCC activity. The ability of any particular polypeptide of the present disclosure to mediate lysis of the target cell by ADCC can be assayed. To assess ADCC activity, a polypeptide of interest (e.g., an antibody) is added to target cells in combination with immune effector cells, resulting in cytolysis of the target cell. Cytolysis is generally detected by the release of label (e.g., radioactive substrates, fluorescent dyes or natural intracellular proteins) from the lysed cells. Useful effector cells for such assays include peripheral blood mononuclear cells (PBMC) and Natural Killer (NK) cells. Specific examples of in vitro ADCC assays are described in Bruggemann et al., J. Exp. Med. 166:1351 (1987); Wilkinson et al., J. Immunol. Methods 258:183 (2001); Patel et al., J. Immunol. Methods 184:29 (1995). Alternatively, or additionally, ADCC activity of the antibody of interest can be assessed in vivo, e.g., in an animal model such as that disclosed in Clynes et al., Proc. Natl. Acad. Sci. USA 95:652 (1998).

As used herein, the terms “condition” and “conditioning” refer to processes by which a patient is prepared for receipt of a transplant, e.g., a transplant containing hematopoietic stem cells. Such procedures promote the engraftment of a hematopoietic stem cell transplant (for instance, as inferred from a sustained increase in the quantity of viable hematopoietic stem cells within a blood sample isolated from a patient following a conditioning procedure and subsequent hematopoietic stem cell transplantation. According to the methods described herein, a patient may be conditioned for hematopoietic stem cell transplant therapy by administration to the patient of an ADC, antibody or antigen-binding fragment thereof capable of binding CD38 expressed by hematopoietic stem cells. As described herein, the antibody may be covalently conjugated to a cytotoxin so as to form a drug-antibody conjugate. Administration of an antibody, antigen-binding fragment thereof, or ADC capable of binding the foregoing antigen to a patient in need of hematopoietic stem cell transplant therapy can promote the engraftment of a hematopoietic stem cell graft, for example, by selectively depleting endogenous hematopoietic stem cells, thereby creating a vacancy filled by an exogenous hematopoietic stem cell transplant.

As used herein, the term “effective amount” or “therapeutically effective amount” refers to an amount of a therapeutic agent, e.g., an anti-CD38 antibody, or an antigen-binding fragment thereof, or an anti-CD38 linked ADC, that is sufficient to achieve the desired result in the context of treating, preventing, ameliorating, or reducing the symptoms of a disease or disorder in a patient. For example, in some embodiments, a therapeutically effective amount of an anti-CD38 antibody or ADC is an amount sufficient to reduce or deplete a population of CD38+ cells in a patient that are implicated in the disease of HS. In some embodiments, cells that are targeted for treatment include: CD38 positive (+) monocytes, B cells, NK cells, and/or endothelial cells, for example, CD38+ circulating NK, Memory B cells, and lesional monocyte-derived macrophages and lesional skin endothelial cells which express CD38. In such embodiments, the therapeutically effective amount can be, for example, an amount sufficient to selectively deplete endogenous CD38 positive immune cells that are implicated in the disease of HS and/or its symptoms from the patient, and/or an amount sufficient to promote the engraftment of a hematopoietic stem cell transplant in the patient. In other embodiments, a therapeutically effective amount of an anti-CD38 antibody or ADC is an amount sufficient to have an effect on an autoimmune disease for example, hidradenitis suppurativa (HS) in a human patient

As used herein, the term “half-life” refers to the time it takes for the plasma concentration of the antibody drug in the body to be reduced by one half or 50% in a subject, e.g., a human subject. This 50% reduction in serum concentration reflects the amount of drug circulating.

As used herein, the phrase “substantially cleared from the blood” refers to a point in time following administration of a therapeutic agent (such as an anti-CD38 antibody, or antigen-binding fragment thereof) to a patient when the concentration of the therapeutic agent in a blood sample isolated from the patient is such that the therapeutic agent is not detectable by conventional means (for instance, such that the therapeutic agent is not detectable above the noise threshold of the device or assay used to detect the therapeutic agent). A variety of techniques known in the art can be used to detect antibodies, or antibody fragments, such as ELISA-based detection assays known in the art or described herein. Additional assays that can be used to detect antibodies, or antibody fragments, include immunoprecipitation techniques and immunoblot assays, among others known in the art.

The terms “specific binding” or “specifically binding”, as used herein, refers to the ability of an antibody to recognize and bind to a specific protein structure (epitope) rather than to proteins generally. If an antibody is specific for epitope “A”, the presence of a molecule containing epitope A (or free, unlabeled A), in a reaction containing labeled “A” and the antibody, will reduce the amount of labeled A bound to the antibody. By way of example, an antibody “binds specifically” to a target if the antibody, when labeled, can be competed away from its target by the corresponding non-labeled antibody. In one embodiment, an antibody specifically binds to a target, e.g., CD38, if the antibody has a K_{D} for the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, or less (less meaning a number that is less than 10⁻¹², e.g. 10⁻¹³). In one embodiment, the term “specific binding to CD38” or “specifically binds to CD38,” as used herein, refers to an antibody or that binds to CD38 and has a dissociation constant (K_{D}) of 1.0×10⁻⁷ M or less, as determined by surface plasmon resonance. In one embodiment, K_{D} (M) is determined according to standard bio-layer interferometery (BLI). In one embodiment, K_{off} (1/s) is determined according to standard bio-layer interferometery (BLI). It shall be understood, however, that the antibody may be capable of specifically binding to two or more antigens which are related in sequence. For example, in one embodiment. an antibody can specifically bind to both human and a non-human (e.g., mouse, cynomolgus or non-human primate) orthologs of CD38. Thus, as used herein, an antibody that “specifically binds to human CD38” is intended to refer to an antibody that binds to human CD38 (and possibly CD38 from one or more non-human species, such as cynomolgus) but does not substantially bind to non-CD38 proteins. Preferably, the antibody binds to human CD38 with a K_{D} of 1×10⁻⁷ M or less, a K_{D} of 5×10⁻⁸ M or less, a K_{D} of 3×10⁻⁸ M or less, a K_{D} of 1×10⁻⁸ M or less, or a K_{D} of 5×10⁻⁹ M or less.

As used herein, the term “human antibody” is intended to include antibodies having variable regions derived from human germline immunoglobulin sequences. In embodiments in which a human antibody contains a constant region, the constant region can likewise be derived from human germline immunoglobulin sequences. A human antibody may include amino acid residues not encoded by human germline immunoglobulin sequences (e.g., mutations introduced by random or site-specific mutagenesis in vitro or during gene rearrangement or by somatic mutation in vivo). However, the term “human antibody”, as used herein, is not intended to include antibodies in which CDR sequences derived from the germline of another mammalian species, such as a mouse, have been grafted onto human framework sequences. A human antibody can be produced in a human cell (for example, by recombinant expression) or by a non-human animal or a prokaryotic or eukaryotic cell that is capable of expressing functionally rearranged human immunoglobulin (such as heavy chain and/or light chain) genes. When a human antibody is a single chain antibody, it can include a linker peptide that is not found in native human antibodies. For example, an Fv can contain a linker peptide, such as two to about eight glycine or other amino acid residues, which connects the variable region of the heavy chain and the variable region of the light chain. Human antibodies can be made by a variety of methods known in the art including phage display methods or yeast display methods using antibody libraries derived from human immunoglobulin sequences. Human antibodies can also be produced using transgenic mice that are incapable of expressing functional endogenous immunoglobulins, but which can express human immunoglobulin genes (see, for example, PCT Publication Nos. WO 1998/24893; WO 1992/01047; WO 1996/34096; WO 1996/33735; U.S. Pat. Nos. 5,413,923; 5,625,126; 5,633,425; 5,569,825; 5,661,016; 5,538,806; 5,814,318; 5,885,793; 5,916,771; and 5,939,598).

The term “chimeric antibody” is intended to refer to antibodies in which the variable region sequences are derived from one species and the constant region sequences are derived from another species, such as an antibody in which the variable region sequences are derived from a rat antibody and the constant region sequences are derived from a human antibody.

“Humanized” forms of non-human (e.g., murine or rat) antibodies are immunoglobulins that contain minimal sequences derived from non-human immunoglobulin. In general, a humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody can also comprise all or a portion of an immunoglobulin constant region (Fc), typically that of a human immunoglobulin consensus sequence. Methods of antibody humanization are known in the art. See, e.g., Riechmann et al., 1988, Nature 332:323-7; U.S. Pat. Nos. 5,530,101; 5,585,089; 5,693,761; 5,693,762; and U.S. Pat. No. 6,180,370 to Queen et al.; EP239400; PCT publication WO 91/09967; U.S. Pat. No. 5,225,539; EP592106; EP519596; Padlan, 1991, Mol. Immunol., 28:489-498; Studnicka et al., 1994, Prot. Eng. 7:805-814; Roguska et al., 1994, Proc. Natl. Acad. Sci. 91:969-973; and U.S. Pat. No. 5,565,332.

The present disclosure describes parenteral administration of an anti-CD38 antagonist to a patient with HS. As used herein, “parenteral administration” of a pharmaceutical composition, comprising an anti-CD38 antagonist, includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intratumoral, and intrasynovial injection or infusions; and kidney dialytic infusion techniques. Regional perfusion is also contemplated. Preferred embodiments may include the intravenous and the subcutaneous routes. Topical compositions comprising an anti-CD38 antagonist, and a carrier suitable for topical administration for the treatment, prevention and amelioration of symptoms associated with HS is contemplated herein. The anti-CD38 antagonist, for example, an anti-CD38 antibody, or an antigen-binding fragment thereof, in the composition of this disclosure is in liquid, or semi-solid form, according to need, and may be administered parenterally or applied topically to the site of HS.

The term “excipient” or “carrier” is used herein to describe any ingredient other than the compound(s) of the present disclosure. The choice of excipients will to a large extent depend on factors such as the particular mode of administration, the effect of the excipient on solubility and stability, and the nature of the dosage form. “Pharmaceutically acceptable excipient” includes any and all solvents, dispersion media, coatings, antibacterial and antifungal agents, isotonic and absorption delaying agents, and the like that are physiologically compatible. Some examples of pharmaceutically acceptable excipients are water, saline, phosphate buffered saline, dextrose, glycerol, ethanol and the like, as well as combinations thereof. In some cases, isotonic agents, for example, sugars, polyalcohols such as mannitol, sorbitol, or sodium chloride will be included in the composition. Additional examples of pharmaceutically acceptable substances are wetting agents or minor amounts of auxiliary substances such as wetting or emulsifying agents, preservatives or buffers, which enhance the shelf life or effectiveness of the antibody. In some embodiments, a pharmaceutical composition of the present disclosure may comprise hyaluronidase (e.g., recombinant human hyaluronidase). In certain embodiments, the pharmaceutical composition comprising hyaluronidase may be for subcutaneous administration.

CD38 was first identified by the herculean task of E. L. Reinherz and S. F. Schlossman to characterize and identify cell surface markers with monoclonal antibodies, as part of their pioneer search of molecules for immunophenotyping of T-cells. However, beyond a role as structural marker, CD38 is involved in several physiological and pathological conditions. In particular, CD38 is the main NAD-degrading enzyme in several mammalian tissues. Some pharmacological approaches to inhibit CD38 take advantage of its role as a cell surface marker when the intention is to target the killing or modulate CD38-positive cancer and/or immune cells.

Human CD38 (hCD38) or as is commonly known: 2′-phospho-ADP-ribosyl cyclase/Z-phospho-cyclic-ADP-ribose transferase, (EC:3.2.2.6) is expressed by the CD38 gene in humans and has a human 300 amino acid protein sequence that is provided in NCBI Reference Sequence: NP_001766.2, and accession no NP 001766, the disclosure of which is incorporated by reference herein in its entirety.

CD38, also known as cyclic ADP ribose hydrolase, is a transmembrane glycoprotein that is highly expressed on MM cells and at low levels on normal lymphoid and myeloid cells. The protein encoded by this gene is a non-lineage-restricted, type II transmembrane glycoprotein that synthesizes and hydrolyzes cyclic adenosine 5′-diphosphate-ribose, an intracellular calcium ion mobilizing messenger. The release of soluble protein and the ability of membrane-bound protein to become internalized indicate both extracellular and intracellular functions for the protein. This protein has an N-terminal cytoplasmic tail, a single membrane-spanning domain, and a C-terminal extracellular region with four N-glycosylation sites. Crystal structure analysis demonstrates that the functional molecule is a dimer, with the central portion containing the catalytic site. It is used as a prognostic marker for patients with chronic lymphocytic leukemia. Alternative splicing results in multiple transcript variants.

There have been many anti-CD38 monoclonal antibodies investigated, with Daratumumab® (IgG1-kappa; fully human) being the most promising and only FDA-approved agent. Isatuximab (SAR650984; IgG1-kappa; chimeric) and MOR202 (IgG1-lambda; fully human) are currently still being investigated.

Anti-CD38 Daratumumab® destroys MM cells through multiple mechanisms. Antibody-dependent cellular cytotoxicity (ADCC) is the cytotoxicity of an antibody-coated target cell by an effector cell via release of cytotoxic granules or by the expression of cell death-inducing molecules. Effector cells include natural killer cells, neutrophils, eosinophils, dendritic cells, monocytes and macrophages. Complement dependent cytotoxicity (CDC) occurs when the binding antibody starts the complement cascade, which results in an attack on the cell membrane causing cell lysis and death. Cell death through antibody-dependent cellular phagocytosis (ADCP) is caused by macrophages. There have been other proposed mechanisms including direct induction of apoptosis and inhibition of CD38 ectoenzyme activity. The most recent data suggested an immune modulatory role of Daratumumab® which included depletion of CD38 T reg, MDSC, and B regs and an observation of increased clonal T cells in responding patients, which suggests the possibility of an adaptive immune mechanism in response.

### Antagonists of CD38 Useful for the Treatment and Prevention of HS

The term “antagonists of CD38” includes agents which inhibit either the expression or activity of CD38, or both. Examples of CD38 inhibitors include, anti-CD38 antibodies or antigen-binding fragments thereof, antisense molecules that target and inhibit CD38 transcription and translation, for example, siRNA molecules that target the mRNA which encodes CD38, and small molecule inhibitors that target CD38, for example, modified N1-Inosine 5-monophosphate (Ni-IMP) inhibitors described in Watt J M, Graeff R, Potter B V L. Small Molecule CD38Inhibitors: Synthesis of 8-Amino-N1-inosine Y-monophosphate. Analogues and Early Structure-Activity Relationship. Molecules. 2021 Nov. 26; 26(23):7165, the disclosure of which is incorporated herein by reference in its entirety, and anti-CD38 antibodies, or antigen-binding fragments thereof conjugated to a cytotoxic agent (i.e. an anti-CD38 antibody drug conjugate or ADC). In various illustrative embodiments, the cytotoxic agent used in the anti-CD38 ADC may be selected from, e.g., a maytansinoid, a small drug, a tomaymycin derivative, a leptomycin derivative, a prodrug, a taxoid, CC-1065 and a CC-1065 analog, or any cytotoxic agent described in U.S. Pat. No. 8,153,765 (which is incorporated by reference in its entirety herein), among others described herein.

### Anti-CD38 Antibodies

In some embodiments, the agent that specifically binds to CD38, described herein is an anti-CD38 antibody or an antigen-binding fragment thereof. In certain embodiments, the anti-CD38 antibody is a human or humanized monoclonal antibody. In some embodiments, the anti-CD38 antibody is an antibody described in U.S. Pat. No. 8,153,765, which is incorporated by reference in its entirety herein. In some embodiments, the anti-CD38 antibody is produced by a hybridoma cell line deposited at the American Type Culture Collection under deposit number PTA-7670. In some embodiments, the anti-CD38 antibody is Ab2, Ab3, Ab4, Daratumumab®, MOR202 (Raab et al., Blood 128:1152 (2016)), HexaBody®-CD38 (Genmab/Janssen), an anti-CD38 SIFbody (Momenta), anti-CD38 monoclonal Ab, isatuximab, and isatuximab-irfc (SARCLISA®) or TSK011010 (CASI). Various of these anti-CD38 antibodies or antigen-binding fragments thereof are commercially available. In some embodiments, the anti-CD38 antibody, or antigen-binding fragment thereof is the antibody Daratumumab® and its antigen-binding fragments thereof. Daratumumab® has been shown to inhibit CD38 activity in various clinical studies, see for example, NCT03277105 to investigate subcutaneous versus (vs.) intravenous administration of Daratumumab® in participants with relapsed or refractory Multiple Myeloma, the disclosure of which is incorporated herein by reference in its entirety. In some embodiments, anti-CD38 antibodies, and their antigen-binding fragments thereof, like, which specifically bind to CD38 and inhibit CD38 activity, such that when administered to a subject suffering from HS, or symptoms related to HS, or is at risk of suffering from a disorder in which CD38 activity is detrimental, the disorder is treated. In one embodiment, a CD38 inhibitor is an anti-CD38 antibody, or an antigen-binding fragment thereof, which inhibits CD38 activity. In another embodiment, the anti-CD38 antibody inhibitors of the present disclosure are used to treat HS, or a symptom of HS or diminish the symptoms associated with HS, as described herein.

The term “antibody,” as used herein, is intended to refer to immunoglobulin molecules comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The antibodies of the invention are described in further detail in U.S. Pat. Nos. 6,090,382, 6,258,562, 6,509,015, and 7,223,394, and in U.S. patent application Ser. No. 10/302,356 (now abandoned), each of which is incorporated herein by reference in its entirety.

The term “antigen-binding fragment” of an antibody (or simply “antibody fragment”). as used herein, refers to one or more fragments of an antibody that retain the ability to specifically bind to an antigen (e.g., hCD38). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′).sub.2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). The antibody portions of the invention are described in further detail in U.S. Pat. Nos. 6,090,382, 6,258,562, 6,509,015; and 7,223,394, and in U.S. patent application Ser. No. 10/302,356 (now abandoned), each of which is incorporated herein by reference in its entirety.

Antigen-binding fragments are produced by recombinant DNA techniques, or by enzymatic or chemical cleavage of intact immunoglobulins. Binding fragments include Fab, Fab′, F(ab′)₂, Fabc, Fv, single chains, and single-chain antibodies. Other than “bispecific” or “bifunctional” immunoglobulins or antibodies, an immunoglobulin or antibody is understood to have each of its binding sites identical. A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor, L. D. et al. (1992) Nucl. Acids Res. 20:6287) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

An “isolated antibody”, as used herein, is intended to refer to an antibody that is substantially free of other antibodies having different antigenic specificities (e.g., an isolated antibody that specifically binds hCD38 is substantially free of antibodies that specifically bind antigens other than hCD38). An isolated antibody that specifically binds hCD38 may, however, have cross-reactivity to other antigens, such as CD38 molecules from other species. Moreover, an isolated antibody may be substantially free of other cellular material and/or chemicals.

A “neutralizing antibody”, as used herein (or an “antibody that neutralized hCD38 activity”), is intended to refer to an antibody whose binding to hCD38 results in inhibition of the biological activity of hCD38. This inhibition of the biological activity of hCD38 can be assessed by measuring one or more indicators of hCD38 biological activity, such as hCD38-induced cytotoxicity (either in vitro or in vivo), hCD38-induced cellular activation and hCD38 binding to hCD38 receptors. These indicators of hCD38 biological activity can be assessed by one or more of several standard in vitro or in vivo assays known in the art (see U.S. Pat. No. 6,090,382). Preferably, the ability of an antibody to neutralize hCD38 activity is assessed by inhibition of hCD38-induced cytotoxicity of L929 cells. As an additional or alternative parameter of hCD38 activity, the ability of an antibody to inhibit hCD38-induced expression of ELAM-1 on HUVEC, as a measure of hCD38-induced cellular activation, can be assessed.

The present disclosure is performed without undue experimentation using, unless otherwise indicated, conventional techniques of molecular biology, microbiology, virology, recombinant DNA technology, solid phase and liquid nucleic acid synthesis, peptide synthesis in solution, solid phase peptide synthesis, immunology, cell culture, and formulation. Such procedures are described, for example, in Sambrook, Fritsch & Maniatis, Molecular Cloning: A Laboratory Manual, Cold Spring Harbor Laboratories, New York, Second Edition (1989), whole of Vols I, II, and III; DNA Cloning: A Practical Approach, Vols. I and II (D. N. Glover, ed., 1985), IRL Press, Oxford, whole of text; Oligonucleotide Synthesis: A Practical Approach (M. J. Gait, ed, 1984) IRL Press, Oxford, whole of text, and particularly the papers therein by Gait, pp 1-22; Atkinson et al, pp 35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984); Methods In Enzymology (S. Colowick and N. Kaplan, eds., Academic Press, Inc.), whole of series; J. F. Ramalho Ortigao, “The Chemistry of Peptide Synthesis” In: Knowledge database of Access to Virtual Laboratory website (Interactiva, Germany); Sakakibara, D., Teichman, J., Lien, E. Land Fenichel, R. L. (1976). Biochem. Biophys. Res. Commun. 73 336-342; Merrifield, R B. (1963). J. Am. Chem. Soc. 85, 2149-2154; Barany, G. and Merrifield, R B. (1979) in The Peptides (Gross, E. and Meienhofer, 3. eds.), vol. 2, pp. 1-284, Academic Press, New York. 12. Wiinsch, E., ed. (1974) Synthese von Peptiden in Houben-Weyls Metoden der Organischen Chemie (Muler, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474; Handbook of Experimental Immunology, Vols. I-IV (D. M. Weir and C. C. Blackwell, eds., 1986, Blackwell Scientific Publications); and Animal Cell Culture: Practical Approach, Third Edition (John R. W. Masters, ed., 2000); each of these references are incorporated herein by reference in their entireties.

As used herein, the term “subject”, “individual” or “patient,” used interchangeably, refers to any animal, including mammals, preferably mice, rats, other rodents, rabbits, dogs, cats, swine, cattle, sheep, horses, or primates, and most preferably humans. In some embodiments, the “subject,” “individual,” or “patient” is in need of said treatment.

In some embodiments, the an anti-CD38 antagonist, e.g., an anti-CD38 antibody, or antigen-binding portion thereof, or an anti-CD38 ADC are administered in a therapeutically effective amount. As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

As used herein, the term “treating” or “treatment” refers to one or more of (1) inhibiting the disease, i.e. HS; for example, inhibiting a disease, condition or disorder in an individual who is experiencing or displaying the pathology or symptomatology of the disease, condition or disorder (i.e., arresting further development of the pathology and/or symptomatology); and (2) ameliorating the disease, i.e. HS; for example, ameliorating a disease, condition or disorder in an individual who is experiencing or displaying the pathology or symptomatology of the disease, condition or disorder (i.e., reversing the pathology and/or symptomatology) such as decreasing the severity of disease. In some embodiments, the administration of an anti-CD38 antagonist to a subject that is likely to develop HS or symptoms of HS and thereby preventing the occurrence of the full disease, i.e. HS condition or disorder in an individual who may be predisposed to the disease, condition or disorder but does not yet experience or display the pathology or symptomatology of the disease. In some embodiments, treating refers to inhibiting or ameliorating the disease HS or its symptoms. As used herein, treating the disease does not mean preventing the disease in a subject that is otherwise asymptomatic.

### Compositions for the Treatment of Hidradenitis Suppurativa

The present disclosure provides compositions, pharmaceutical compositions, methods and articles of manufacture for the treatment of HS in a subject in need of treatment.

When employed as pharmaceuticals, the anti-CD38 antagonists of the invention can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

The anti-CD38 antagonist(s) may be effective over a wide dosage range and is generally administered in a pharmaceutically effective amount. It will be understood, however, that the amount of the anti-CD38 antagonist actually administered will usually be determined by a physician, according to the relevant circumstances, including the condition to be treated, the chosen route of administration, the actual anti-CD38 antagonist administered, the age, weight, and response of the individual patient, the severity of the patient's symptoms, and the like.

The anti-CD38 antagonist, e.g., an anti-CD38 antibody, or antigen-binding portion thereof, or an anti-CD38 ADC, and other CD38 antagonists of the invention, can be incorporated into pharmaceutical compositions suitable for administration to a subject. Typically, the pharmaceutical composition comprises an anti-CD38 antibody, or antigen-binding fragment thereof of the invention and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it is preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody, antibody portion

For preparing liquid compositions such as a solution of an anti-CD38 antibody, or antigen-binding fragment thereof, the anti-CD38 antagonist is mixed with a pharmaceutical excipient to form a liquid preformulation composition containing a homogeneous mixture of a CD38 antagonist agent of the present application. When referring to these preformulation compositions as homogeneous, the anti-CD38 antagonist active ingredient is typically dispersed evenly throughout the composition so that the composition can be readily subdivided into equally effective unit dosage forms such as injections, infusions, emulsions and other parenteral and transdermal and dermal or topical forms. This liquid preformulation is then subdivided into unit dosage forms of the type described above containing from, for example, about 0.1 mg to about 3,000 mg of the active anti-CD38 antagonist ingredient of the present disclosure.

Methods for Preventing and/or Treating Hidradenitis Suppurativa

Methods of the present disclosure include administering a therapeutically effective amount of a CD38 antagonist or inhibitor active agent to a subject in need thereof, i.e. a subject diagnosed with HS, or a subject likely to develop HS. In various embodiments, a subject to be treated for HS is a mammal subject, a laboratory animal, a non-human primate, or humans, preferably the subject is a human subject in need of treatment against HS. In various embodiments, the anti-CD38 antagonist is an anti-CD38 antibody, or antigen-binding fragment thereof, or an anti-CD38 antibody, or antigen-binding fragment thereof conjugated to a toxic agent (anti-CD38 ADC). In various embodiments, the anti-CD38 antagonist, is preferably a human anti-CD38 antibody or antigen-binding fragment thereof formulated in a pharmaceutical composition, with pharmaceutical excipients, diluents or carrier substances for administration to the subject in need thereof using parenteral or topical administration routes.

Initially, patients are diagnosed using established methodologies. For example, Hidradenitis suppurativa begins where skin touches skin. HS tends to begin in an area with thick, coarse hair like the armpits and groin. As the disease progresses, some people may see HS under their breasts. HS can also develop in less common locations like near an ear or around the belly button. A few people have developed HS on their face, neck, or back, but this is rare. HS patients may notice breakouts on their skin that look like pimples or boils. Often, these breakouts clear for a while. Later, patients sometimes experience new breakouts develop in the same area. Sometimes, the breakouts develop in exactly the same spot. Other symptoms may include some discomfort in the areas where the bumps or pimples exist. The area where the lump will appear may swell. Some people say their skin burns, itches, or sweats excessively. In some examples, HS patients' first sign is often a painful spot that looks like a deep pimple, acne cyst, or boil. This spot often appears on an armpit (as shown here) or in the groin area. Some people develop a spot on their buttocks or inner thigh. As HS progresses, you see more lumps that may look like acne or boils. These lumps can grow and join together. As the lumps grow together, they fill up with fluid and become painful. These painful lumps are called abscesses. Large, painful abscess break open in some cases. In the advanced stages, some people see small black bumps that look like blackheads. These spots often appear in twos. Abscesses heal slowly (if at all) and return; scars form. The repetitive healing and reopening eventually cause tunnels to form beneath the skin, which are called sinus tracts, and permanent scars. Some people have HS wounds on their skin all the time. For others, scars may be the only sign of HS for a while. When an abscess breaks open, blood and pus spill out. Diagnosis of HS is well known in the art and can be further illustrated in: Alikhan A, Sayed C, et al. “North American clinical management guidelines for hidradenitis suppurativa: A publication from the United States and Canadian Hidradenitis Suppurativa Foundations Part I: Diagnosis. evaluation, and the use of complementary and procedural management.” J Am Acad Dermatol 2019; 81:76-90, the disclosure of which is incorporated herein by reference in its entirety.

The methods of treatment primarily focuses on treating HS by reducing the cell numbers and blocking activity of CD38 positive immune and other cells that are implicated in the disease of HS. Without being bound by any particular theory, it is believed that the anti-CD38 antagonist reduces the numbers and/or blocking activity of CD38 positive immune cells and other endogenous cells that are pathogenic and are implicated in the pathogenesis and drivers of HS. For example, in some embodiments, a therapeutically effective amount of an anti-CD38 antagonist an amount sufficient to reduce or deplete a population of CD38+ cells in a patient that are implicated in the disease of HS. In some embodiments, cells that are targeted for treatment include: one or more of the following CD38 positive (+) cell types: monocytes, B cells, NK cells, and endothelial cells, for example, CD38+ circulating NK, Memory B cells, skin-tropic intermediate monocytes, lesional monocyte-derived macrophages, and lesional skin endothelial cells, all of which express CD38 on the surface of the cells.

The term “dosing,” as used herein, refers to the administration of an anti-CD38 antagonist (e.g., an anti-CD38 antibody, or an antigen-binding fragment thereof) to achieve a therapeutic objective (e.g., treatment of HS, or amelioration of symptoms of HS).

A “dosing regimen” describes a treatment schedule for an anti-CD38 antagonist (e.g., an anti-CD38 antibody, or an antigen-binding portion thereof), e.g., a treatment schedule over a prolonged period of time or throughout the course of treatment, e.g. administering a first dose of a an anti-CD38 antagonist (e.g., an anti-CD38 antibody, or an antigen-binding portion thereof) at week 0 followed by a second dose of a an anti-CD38 antagonist (e.g., an anti-CD38 antibody, or an antigen-binding portion thereof) on a weekly or biweekly dosing regimen. The anti-CD38 antagonist(s) dosed during the regiment, can be the same or different.

The term “multiple-variable dose” includes different doses of an anti-CD38 antagonist (e.g., an anti-CD38 antibody, or an antigen-binding portion thereof) which are administered to a subject for therapeutic treatment “Multiple-variable dose regimen” or “multiple-variable dose therapy” describes a treatment schedule which is based on administering different amounts of an anti-CD38 antagonist (e.g., an anti-CD38 antibody, or an antigen-binding portion thereof) at various time points throughout the course of treatment

The term “induction dose” or “loading dose,” used interchangeably herein, refers to the first dose(s) of an anti-CD38 antagonist (e.g., an anti-CD38 antibody, or an antigen-binding portion thereof, or an anti-CD38 antibody, or antigen-binding fragment thereof conjugated to a cytotoxic agent) which is initially used to treat hidradenitis suppurativa (HS). The loading dose may be larger in comparison to the subsequent maintenance or treatment dose. The induction dose can be a single dose or, alternatively, a set of doses. For a purely illustrative example only, a 160 mg dose may be administered as a single 160 mg dose, as two doses of 80 mg each, or four doses of 40 mg each. In one embodiment, an induction dose is subsequently followed by administration of smaller doses of an anti-CD38 antagonist (e.g., an anti-CD38 antibody, or an antigen-binding portion thereof), e.g., the treatment or maintenance dose(s). The induction dose is administered during the induction or loading phase of therapy. In one embodiment of the invention, the induction dose is at least twice the given amount of the treatment dose. In one embodiment of the invention, the induction dose when dosed intravenously ranges from 1 to 100 mg/kg (mg/kg body weight of the subject) dosed once weekly, every two weeks. every three weeks or dosed every four weeks or combinations thereof. An equivalent dose when dosed subcutaneously, could include a dose ranging from about 100 mg to about 3,000 mg per mL, in a final volume ranging from about 0.5 mL to about 1,000 mL, dosed once weekly, every two weeks, every three weeks or dosed every four weeks, or combinations thereof. In one embodiment of the invention, the induction dose comprise a dose ranging from about 100 mg dose to about 3,000 mg followed by a dose ranging from about 100 mg dose to about 2,000 mg, wherein the two induction doses are administered 1 to 4 weeks apart. In some embodiments, intravenous infusion of an anti-CD38 antagonist at a dose of about 1 to 100 milligrams per kilogram (mg/kg) once weekly in Cycle 1 and 2. every 2 weeks in Cycle 3 to 6, every 4 weeks thereafter wherein the duration for each cycle is about 28 days. In some embodiments, a subject with HS is administered an anti-CD38 antagonist at a dose from about 100 mg to about 3,000 mg, for example, 1800 mg via subcutaneous injection, once weekly in Cycle 1 and 2, every 2 weeks in Cycle 3 to 6, every 4 weeks thereafter wherein the duration for each cycle is about 28 days.

The term “maintenance therapy” or “maintenance dosing regimen” refers to a treatment schedule for a subject or patient diagnosed with HS, to enable them to maintain their health in a given state, e.g., reduced number of inflammatory lesions or achieving a clinical response. In one embodiment, a maintenance therapy of the invention is used for a subject or patient diagnosed with a disorder/disease, e.g., HS to enable them to maintain their health in a state which is completely free of symptoms or a reduction in symptoms associated with the disease. In one embodiment, a maintenance therapy of the invention is used for a subject or patient diagnosed with a HS, to enable them to maintain their health in a state which is substantially free of symptoms associated with the disease. In one embodiment, a maintenance therapy of the invention is used for a subject or patient diagnosed with a HS, to enable them to maintain their health in a state where there is a significant reduction in symptoms associated with the disease.

The term “treatment phase” or “maintenance phase,” as used herein, refers to a period of treatment comprising administration of an anti-CD38 antagonist, such as an anti-CD38 antibody, or an antigen-binding fragment thereof, or an anti-CD38 ADC to a subject in order to maintain a desired therapeutic effect, e.g., improved symptoms associated with HS.

The term “maintenance dose” or “treatment dose” is the amount of an anti-CD38 antagonist, such as an anti-CD38 antibody, or an antigen-binding fragment thereof, or an anti-CD38 ADC, taken by a subject to maintain or continue a desired therapeutic effect. A maintenance dose can be a single dose or, alternatively, a set of doses. A maintenance dose is administered during the treatment or maintenance phase of therapy. In one embodiment, a maintenance dose(s) is smaller than the induction dose(s) and may be equal to each other when administered in succession. In one embodiment, the invention provides a maintenance dose of 20 mg to 400 mg of a CD38 inhibitor, for example, an anti-CD38 antibody, or antigen-binding fragment thereof, administered intravenously, or subcutaneously to a subject weekly or biweekly. In one embodiment, the maintenance dose is administered every week or every other week beginning 1 of 2 weeks after the last loading dose. In one embodiment, a maintenance dose is administered about 4 weeks following the initial loading dose.

The terms “biweekly dosing regimen,” “biweekly dosing,” and “biweekly administration,” as used herein, refer to the time course of administering a substance (e.g., an anti-CD38 antagonist, such as an anti-CD38 antibody, or an antigen-binding fragment thereof, or an anti-CD38 ADC) to a subject to achieve a therapeutic objective, e.g., throughout the course of treatment. The biweekly dosing regimen is not intended to include a weekly dosing regimen. Preferably, the substance is administered every 9-19 days, or 10-18 days, more preferably, every 11-17 days, or 12-16 days, even more preferably, every 13-15 days, and most preferably, every 14 days. In one embodiment, the biweekly dosing regimen is initiated in a subject at week 0 of treatment. In another embodiment, a maintenance dose is administered on a biweekly dosing regimen. In one embodiment, both the loading and maintenance doses are administered according to a biweekly dosing regimen. In one embodiment, biweekly dosing includes a dosing regimen where doses of an anti-CD38 antagonist, such as an anti-CD38 antibody, or an antigen-binding fragment thereof, or an anti-CD38 ADC are administered to a subject every other week consecutively for a given time period, e.g., 4 weeks, 8 weeks, 16, weeks, 24 weeks, 26 weeks, 32 weeks, 36 weeks, 42 weeks, 48 weeks, 52 weeks, 56 weeks, etc. Biweekly dosing methods are also described in US 20030235585, incorporated by reference herein. Biweekly administration is also referred to as “eow” or “every other week”.

The terms “qwk,” “qw,” or “ew,” as used interchangeably herein, refer to a weekly dosing regimen, where a substance (e.g., a human anti-CD38 antibody, or an antigen-binding portion thereof) is administered to a subject once a week (or every week) to achieve a therapeutic objective, e.g., treating HS. A “weekly dosing regimen” as used herein, refers to the time course of administering a substance (e.g., an anti-CD38 antibody) to a subject to achieve a therapeutic objective, e.g., throughout the course of treatment. Weekly administration is more frequent than biweekly, e.g, every 6-8 days, every 5-8 days, or every 7 days.

The term “fixed dose” or “total body dose” refers to a dose which is a constant amount delivered with each administration and is not dependent on the weight of the subject being treated. The term “fixed dose” dose not include weight-based dosing, i.e., mg/kg dosing determinations. In one embodiment, a human CD38 antibody, or antigen-binding portion thereof, is administered to the subject at a fixed dose ranging from 10-500 mg, or from 20-300 mg. In one embodiment, a human CD38 antibody, or antigen-binding portion thereof, is administered to the subject in a fixed dose of 30 mg, 35 mg, 40 mg, 38 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 138 mg, 150 mg, 155 mg, 160 mg, 165 mg, 170 mg, 175 mg, 180 mg, 200 mg, 225 mg, 250 mg, 275 mg, 300 mg, 325 mg, 350 mg, 375 mg, 400 mg, 425 mg, 380 mg, 475 mg, or 500 mg and all intervals therebetween. Ranges of values between any of the aforementioned recited values are also intended to be included in the scope of the invention, e.g., 31 mg, 32 mg, 33 mg, 34 mg, 35 mg, 36 mg, 37 mg, 38 mg, 39 mg, 40 mg, 41 mg, 42 mg, 43 mg, 44 mg, 38 mg, 46 mg, 47 mg, 48 mg, 49 mg, 85 mg, 95 mg, as are ranges based on the aforementioned doses, e.g., 30-50 mg, 20-80 mg, 20-70 mg, 20-60 mg, and 20-50 mg.

In one embodiment, a human CD38 antibody, or antigen-binding portion thereof, is administered to the subject at a fixed dose ranging from 10-5,000 mg, or from 20-3,000 mg. In one embodiment, a human CD38 antibody, or antigen-binding portion thereof, is administered to the subject in a fixed dose of 300 mg, 350 mg, 400 mg, 380 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1380 mg, 1500 mg, 1550 mg, 1600 mg, 1650 mg, 1700 mg, 1750 mg, 1800 mg, 2000 mg, 2250 mg, 2500 mg, 2750 mg, 3000 mg, 3250 mg, 3500 mg, 3750 mg, 4000 mg, 4250 mg, 3800 mg, 4750 mg, or 5000 mg and all intervals therebetween. The fixed doses described above may also be a therapeutically effective amount” or a “prophylactically effective amount dose, or a daily dose. Ranges of values between any of the aforementioned recited values are also intended to be included in the scope of the invention, e.g., 31 mg, 32 mg, 33 mg, 34 mg, 35 mg, 36 mg, 37 mg, 38 mg, 39 mg, 40 mg, 41 mg, 42 mg, 43 mg, 44 mg, 38 mg, 46 mg, 47 mg, 48 mg, 49 mg, 85 mg, 95 mg, as are ranges based on the aforementioned doses, e.g., 30-50 mg, 20-80 mg, 20-70 mg, 20-60 mg, and 20-50 mg, or 310 mg, 320 mg, 330 mg, 340 mg, 350 mg, 360 mg, 370 mg, 380 mg, 390 mg, 400 mg, 410 mg, 420 mg, 430 mg, 440 mg, 380 mg, 460 mg, 470 mg, 480 mg, 490 mg, 850 mg, 950 mg, as are ranges based on the aforementioned doses, e.g., 300-5000 mg, 20-3800 mg, 20-4700 mg, 20-2600 mg, 1500-2,000, and 200-5000 mg.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the anti-CD38 antagonist, for example, anti-CD38 antibody, antibody portion, or other anti-CD38 antagonists may vary according to factors such as the disease state, age, sex, and weight of the individual, and the ability of the antibody, antibody portion, other CD38 inhibitor to elicit a desired response in the individual. A therapeutically effective amount is also one in which any toxic or detrimental effects of the anti-CD38 antagonist e.g. antibody, antibody portion, or other anti-CD38 antagonist are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The present disclosure also provides packaged pharmaceutical compositions or kits for administering the anti-CD38 antagonist or inhibitor, for example, anti-CD38 antibodies, or antigen-binding fragments thereof, of the disclosure for the treatment of HS. In one embodiment of the invention, the kit comprises at least one anti-CD38 antagonist, such as an antibody, and instructions for administration of the anti-CD38 antagonist for treatment of HS. The instructions may describe how, e.g., intravenously, or subcutaneously, or intradermally, or dermally, or topically, dosing frequencies, and when, e.g., at week 0, week 2, week 4, etc., the different doses of the anti-CD38 antagonist (e.g., an anti-CD38 antibody, or an antigen-binding portion thereof) shall be administered to a subject for treatment.

Another aspect of the invention pertains to kits containing a pharmaceutical composition comprising an anti-CD38 antagonist, such as an anti-CD38 antibody, or an antigen-binding fragment thereof, or an anti-CD38 ADC, and a pharmaceutically acceptable carrier and one or more pharmaceutical compositions each comprising optionally an additional therapeutic agent useful for treating hidradenitis suppurativa, and a pharmaceutically acceptable carrier. Alternatively, the kit comprises a single pharmaceutical composition comprising an anti-CD38 antibody, and optionally a combined secondary active agent including one or more medicaments, or drugs useful for treating HS, and a pharmaceutically acceptable carrier. The instructions may describe how, e.g., subcutaneously, intravenously, intradermally, transdermally or topically the compositions of the present invention may be applied, and dosed, and when, e.g., at week 0, week 2, week 4, etc., the different doses of the anti-CD38 antagonist (e.g., an anti-CD38 antibody, or an antigen-binding portion thereof) and/or an optional additional therapeutic agent shall be administered to a subject for treatment of HS.

The kit may contain instructions for dosing of the pharmaceutical compositions for the treatment of HS.

The package or kit alternatively can contain the anti-CD38 antagonist (e.g., an anti-CD38 antibody, or an antigen-binding portion thereof, or an anti-CD38 ADC) and it can be promoted for use, either within the package or through accompanying information, for the uses or treatment of HS symptoms and disease described herein. The packaged pharmaceuticals or kits further can optionally include a second active agent for example, an antibiotic and/or an anti-inflammatory agent, for example, an anti-IL17 antibody packaged with or co-promoted with instructions for using the second agent with a first anti-CD38 antagonist agent (as described herein).

Certain subtypes of HS may be treated in accordance with the invention. In one embodiment, mild, and moderate to severe HS, is treated by administering an anti-CD38 antagonist, e.g., antibody, or antigen-binding portion thereof, or an anti-CD38 ADC, to a subject suffering therefrom. In one embodiment, chronic HS, e.g., mild, and moderate to severe chronic HS, is treated by administering an anti-CD38 antagonist, e.g., an anti-CD38 antibody, or antigen-binding portion thereof, or an anti-CD38 ADC, to a subject suffering therefrom.

The present disclosure also provides a method for treating certain subpopulations of HS patients who may be especially difficult to treat (described in more detail below). For example, in one embodiment, the invention provides a method for treating patients who have a subtherapeutic response to a therapy, such as those who have been unresponsive or intolerant to oral antibiotics for treatment for their HS.

The invention also provides methods for improving HS symptoms in a subject based on indices used to measure the disease state.

Treatment of HS using an anti-CD38 antagonist, e.g., an anti-CD38 antibody, or antigen-binding portion thereof, or an anti-CD38 ADC, may also be determined using measures known in the art. Treatment of HS may be determined using any of the measures described herein, e.g., improvement in Hurley Staging or the Sartorius scale, or any measure known to those in the art.

For example, in one embodiment, an improvement in the Hurley stage of the subject having HS, or any of the measures described herein, is evidence of effective HS treatment In one embodiment, the severity of HS is determined according to the Hurley staging system. Hurley staging is based on assigning the subject having HS one of three different “Stages” depending on the disease level. More specifically, Stage I refers to abscess formation, single or multiple, without sinus tracts and cicatrisation; Stage II refers to recurrent abscesses with tract formation and cicatrisation, as well as single or multiple, widely separated lesions; and Stage III, which refers to diffuse or near-diffuse involvement, or multiple interconnected tracts and abscesses across the entire area. Hurley Stage III is the most severe form. In one embodiment, the subject having HS has HS lesions that are present in at least two distinct anatomic areas (e.g. left and right axilla; or left axilla and left inguinal-crural fold), one of which is at least Hurley Stage II. In another embodiment, the subject being treated has at least one lesion that is at least a Hurley Stage II.

In one embodiment, treatment of HS with an anti-CD38 antagonist, e.g., an anti-CD38 antibody, or antigen-binding portion thereof, or an anti-CD38 ADC, is determined by an improved Hurley score relative to a given baseline, e.g., the Hurley stage of the subject prior to treatment with the anti-CD38 antagonist, e.g., an anti-CD38 antibody, or antigen-binding portion thereof, or an anti-CD38 ADC. In one embodiment, improvement in a Hurley score indicates that the Hurley score of the subject has either improved or been maintained following treatment with an anti-CD38 antagonist, e.g., an anti-CD38 antibody, or antigen-binding portion thereof, or an anti-CD38 ADC. Severity of HS may be determined according to standard clinical definitions. See, for example, Hurley staging (III vs. (I or II)) for HS (Poli F, Jemec G B E, Revuz J., Clinical Presentation. In: Jemec G B E, Revuz J, Leyden J J, editors. HS. Springer, New York, 2006, pp 11-24, the disclosure of which is incorporated herein by reference in its entirety). Hurley stage III disease is the most severe stage of HS, reflecting diffuse or near-diffuse involvement of affected areas.

In one embodiment, the Sartorius scale may be used as an index for measuring efficacy of an anti-CD38 antagonist, e.g., an anti-CD38 antibody, or antigen-binding portion thereof, or an anti-CD38 ADC, for treating HS. The Sartorius scale is described by Sartorius et al. in British Journal of Dermatology, 149: 211-213 (incorporated herein by reference). Briefly, the following outcome variables are explicitly mentioned in reports based on the Sartorius scale: (1) anatomical region involved (axilla, groin, gluteal or other region or inframammary region left and/or right: 3 points per region involved); (2) number and scores of lesions (abscesses, nodules, fistulas, scars: points per lesion of all regions involved: nodules 2; fistulas 4; scars 1; others 1); (3) the longest distance between two relevant lesions, i.e., nodules and fistulas, in each region, or size if only one lesion (<5 cm, 2; <10 cm, 4; >10 cm, 8); and (4) are all lesions clearly separated by normal skin? In each region (yes 0/no 6). By assigning numerical scores to these variables, disease intensity can be quantified in a more clinically meaningful way on an open-ended scale. A total score as well as scores of selected regions chosen for surgical or other intervention can be calculated and followed over time.

In one embodiment, treatment of HS with an anti-CD38 antagonist, e.g., an anti-CD38 antibody, or antigen-binding portion thereof, or an anti-CD38 ADC, is determined according to an achieving an HiSCR (HS Clinical Response) of the subject being treated. The HisSCR is defined as at least a 50% reduction in the total inflammatory lesion (abscess and inflammatory nodule) count (AN count) in a subject relative to baseline, with no increase in abscess count and no increase in draining fistula count. In one embodiment, treatment of HS in a subject is defined as an at least 50% reduction in the inflammatory lesion (abscess and nodule) count.

The HiSCR scoring system was designed to assess HS activity in an affected subject before and after a treatment.

In another embodiment, treatment of HS with an anti-CD38 antagonist, e.g., an anti-CD38 antibody, or antigen-binding portion thereof, or an anti-CD38 ADC, is defined as achieving an HS Physician's Global Assessment (HS-PGA) score, or HS-PGA score, as defined below, of clear (0), minimal (1), or mild (2), with an improvement (i.e., reduction) from baseline HS-PGA score of at least 2 grades, optionally, at the end of a treatment period (such as week 16). The baseline HS-PGA score is the HS-PGA score measured just prior to the commencement of treatment, to which the HS-PGA score obtained after a period of treatment is compared. Both the baseline HS-PGA score and the HS-PGA score obtained after a treatment period are assessed based on the following system and criteria:

### HS-PGA Scoring System

Score Rating Description: 0—Clear No abscesses, no draining fistulas, no nodules; 1—Minimal No abscesses, no draining fistulas, no inflammatory nodules, presence of non-inflammatory nodules; 2—Mild No abscesses or draining fistulas, and less than 5 inflammatory nodules, or Single abscess or draining fistula, and no inflammatory nodules; 3—Moderate No abscesses or draining fistulas, and at least 5 inflammatory nodules, or Single abscess or draining fistula in the presence of inflammatory nodules, or Between 2 and 5 abscesses or draining fistulas with or without inflammatory nodules, up to 10; 4—Severe Between 2 and 5 abscesses and draining fistulas with or without inflammatory nodules that are greater than 10; 5—Very severe More than 5 abscesses or draining fistulas.

The HS-PGA scoring system was designed for use in assessing HS activity before and after a treatment. It is a six-point score that partly depends on the presence/absence of abscesses, draining fistulas, and/or nodules (inflammatory or non-inflammatory), and, if present, the extent of such presence.

The invention also includes a method of decreasing an HS-PGA score of a subject comprising administering an anti-CD38 antagonist, e.g., an anti-CD38 antibody, or antigen-binding portion thereof, or an anti-CD38 ADC, to the subject, such that partial remission of HS is induced. In one embodiment, the invention provides an improvement of at least about 2 grades in the HS-PGA score of a subject having HS, by administering a therapeutically effective amount of an anti-CD38 antagonist, e.g., an anti-CD38 antibody, or antigen-binding portion thereof, or an anti-CD38 ADC to the HS subject in need thereof.

Methods of treatment described herein may include administration of an anti-CD38 antagonist, e.g., an anti-CD38 antibody, or antigen-binding portion thereof, or an anti-CD38 ADC, to a subject to achieve a therapeutic goal, e.g., treatment of HS, or achievement of a clinical response as defined herein. Also included in the scope of the invention are uses of an anti-CD38 antagonist, e.g., an anti-CD38 antibody, or antigen-binding portion thereof, or an anti-CD38 ADC, in the manufacture of a medicament to achieve a therapeutic goal, e.g., treatment, of HS, increase in clinical response as defined herein, and/or maintenance or improvement (reduction) of a HiSCR. Thus, where methods are described herein, it is also intended to be part of this invention that the use of the CD38 inhibitor in the manufacture of a medicament for the purpose of the method is also considered within the scope of the invention. Likewise, where a use of a CD38 inhibitor, e.g., an anti-CD38 antibody, or antigen-binding fragment thereof, in the manufacture of a medicament for the purpose of achieving a therapeutic goal is described, methods of treatment resulting in the therapeutic goal are also intended to be part of the invention.

In some embodiments, the present disclosure provides a use of a composition comprising an anti-CD38 antagonist for the treatment of Hidradenitis suppurativa (HS) in a subject in need thereof. In some embodiments, the anti-CD38 antagonist comprises an anti-CD38 antibody, or an antigen binding fragment thereof, or an anti-CD38 antibody conjugated to a cytotoxic agent. In further examples, the anti-CD38 antibody, or an antigen binding fragment thereof is a human or humanized anti-CD38 antibody, or an antigen binding fragment thereof. For example, the present disclosure provides the use of an anti-CD38 antibody selected from Daratumumab® or an antigen-binding fragment thereof, or SARCLISA® (Sanofi; isatuximab-irfc) or an antigen-binding fragment thereof for the treatment of HS or a symptom related thereto. In some embodiments, the anti-CD38 antibody is SARCLISA® (Sanofi; isatuximab-irfc) or an antigen-binding fragment thereof. In other embodiments, the anti-CD38 antibody is Daratumumab® or an antigen-binding fragment thereof.

In some embodiments, the anti-CD38 antagonist, e.g., an anti-CD38 antibody, or antigen-binding portion thereof, or an anti-CD38 ADC, is systemically administered via intravenous injection, or infusion, or subcutaneously (SC) administered at a dose of about 10 mg-about 5,000 mg, e.g., about 10 mg, 25 mg, 50 mg, 75 mg, 100 mg, 200 mg, 300 mg, 400 mg, 500 mg, 600 mg, 700 mg, 800 mg, 900 mg, or about 1,000 mg, or about 1,200 mg, or about 1,400 mg, or about 1,500 mg, or about 1,600 mg, or about 1,700 mg, or about 1,800 mg, or about 1,900 mg, or about 2,000 mg, or about 2,200 mg, or about 2,400 mg, or about 2,600 mg, or about 2,800 mg, or about 3,000 mg, or about 3,500 mg, or about 4,000 mg or about 4,500 mg, or about 5,000 mg and all intervals and integers therebetween, (e.g., from about 10 mg to about 5,000 mg, and all intervals and integers therebetween). In various embodiments, the dose ranging from 10 mg-about 1,000 mg, e.g., about 10 mg. 25 mg, 50 mg, 75 mg, 100 mg, 200 mg, 300 mg, 400 mg, 500 mg, 600 mg, 700 mg, 800 mg, 900 mg, 1,000 mg, 1,200 mg, 1,400 mg, 1,500 mg, 1,600 mg, 1,700 mg, 1,800 mg, 1,900 mg, 2,000 mg, 2,200 mg, 2,400 mg, 2,600 mg, 2,800 mg, or about 3,000 mg, (e.g., from about 10 mg to about 3,000 mg, and all intervals and integers therebetween) is a daily dose, a weekly dose, a biweekly dose, a monthly dose, a therapeutically effective dose, and combinations thereof.

In some embodiments, the anti-CD38 antagonist, e.g., an anti-CD38 antibody, or antigen-binding portion thereof, or an anti-CD38 ADC, such as Daratumumab®) is administered subcutaneously using an induction regimen, followed by a maintenance regimen. In some embodiments, the induction regimen comprises weekly administration and the maintenance regimen comprises administration every two weeks or every four weeks. In some embodiments, the CD38 antagonist (e.g., CD38 antibody or antigen-binding fragment thereof, such as Daratumumab®) is administered SC at a dose of about 10 mg to about 5,000 mg, e.g., from about 10 mg to about 3,000 mg, at week 0, 1, 2, 3, and 4 and then every two weeks thereafter. In some embodiments, the CD38 antagonist (e.g., CD38 antibody or antigen-binding fragment thereof) is administered subcutaneously, (SC) at a dose of about 20 mg to about 2,000 mg, e.g., 200-2,000 mg, at week 0, 1, 2, 3, and 4 and then every four weeks thereafter. In some embodiments, the CD38 antagonist (e.g., an anti-CD38 antagonist, e.g., an anti-CD38 antibody, or antigen-binding portion thereof, or an anti-CD38 ADC) is administered SC at a dose of about 10 mg to about 5,000 mg (e.g., 50-3,000 mg, and all intervals and integers therebetween) at week 0, 1, 2, 3, and 4 and then every four weeks (monthly) thereafter. In various embodiments, the anti-CD38 antibody, or antigen-binding fragment thereof described in the various methods and dose regimen described above is Daratumumab®.

In some embodiments, the anti-CD38 antagonist, e.g., an anti-CD38 antibody, or antigen-binding portion thereof, or an anti-CD38 ADC, or a pharmaceutical composition comprising such CD38 antagonist, is administered with a loading dose prior to the specified subcutaneous dose. For example, the loading dose can be a high subcutaneous dose (e.g., about 200 mg to about 5,000 mg) with a shorter dosing interval (e.g., every week or every three days) for a few weeks (e.g., two, three, or four weeks) to a few months (e.g., one, two, or three months). In some embodiments, the loading dose is a dose between 200-5,000 mg subcutaneously every week for a few weeks (e.g., two, three, or four weeks) to a few months (e.g., one, two, or three months). In some embodiments, the loading dose is 900 mg subcutaneously every week for a few weeks (e.g., two, three, or four weeks) to a few months (e.g., one, two, or three months). In some embodiments, the loading dose is 1000 mg subcutaneously every week for a few weeks (e.g., two, three, or four weeks) to a few months (e.g., one, two, or three months). In some embodiments, the loading dose is 1100 mg subcutaneously every week for a few weeks (e.g., two, three, or four weeks) to a few months (e.g., one, two, or three months). In some embodiments, the loading dose is 1200 mg subcutaneously every week for a few weeks (e.g., two, three, or four weeks) to a few months (e.g., one, two, or three months). In some embodiments, the loading dose is 1300 mg subcutaneously every week for a few weeks (e.g., two, three, or four weeks) to a few months (e.g., one, two, or three months). In some embodiments, the loading dose is 1400 mg subcutaneously every week for a few weeks (e.g., two, three, or four weeks) to a few months (e.g., one, two, or three months). In some embodiments, the loading dose is 1500 mg subcutaneously every week for a few weeks (e.g., two, three, or four weeks) to a few months (e.g., one, two, or three months). In some embodiments, the loading dose is 1600 mg subcutaneously every week for a few weeks (e.g., two, three, or four weeks) to a few months (e.g., one, two, or three months). In some embodiments, the loading dose is 1700 mg subcutaneously every week for a few weeks (e.g., two, three, or four weeks) to a few months (e.g., one, two, or three months). In some embodiments, the loading dose is 1800 mg subcutaneously every week for a few weeks (e.g., two, three, or four weeks) to a few months (e.g., one, two, or three months). In some embodiments, the loading dose is 1900 mg subcutaneously every week for a few weeks (e.g., two, three, or four weeks) to a few months (e.g., one, two, or three months). In some embodiments, the loading dose is 2000 mg subcutaneously every week for a few weeks (e.g., two, three, or four weeks) to a few months (e.g., one, two, or three months). In some embodiments, the loading dose is 2200 mg subcutaneously every week for a few weeks (e.g., two, three, or four weeks) to a few months (e.g., one, two, or three months). In some embodiments, the loading dose is 2400 mg subcutaneously every week for a few weeks (e.g., two, three, or four weeks) to a few months (e.g., one, two, or three months). In some embodiments, the loading dose is 2600 mg subcutaneously every week for a few weeks (e.g., two, three, or four weeks) to a few months (e.g., one, two, or three months). In some embodiments, the loading dose is 2800 mg subcutaneously every week for a few weeks (e.g., two, three, or four weeks) to a few months (e.g., one, two, or three months).

In some embodiments, the loading dose is 900-3,000 mg subcutaneously every three days for a few weeks (e.g., two, three, or four weeks) to a few months (e.g., one, two, or three months). In some embodiments, the loading dose is 1000 mg subcutaneously every three days for a few weeks (e.g., two, three, or four weeks) to a few months (e.g., one, two, or three months). In some embodiments, the loading dose is 1100 mg subcutaneously every three days for a few weeks (e.g., two, three, or four weeks) to a few months (e.g., one, two, or three months). In some embodiments, the loading dose is 1200 mg subcutaneously every three days for a few weeks (e.g., two, three, or four weeks) to a few months (e.g., one, two, or three months). In some embodiments, the loading dose is 1300 mg subcutaneously every three days for a few weeks (e.g., two, three, or four weeks) to a few months (e.g., one, two, or three months). In some embodiments, the loading dose is 1400 mg subcutaneously every three days for a few weeks (e.g., two, three, or four weeks) to a few months (e.g., one, two, or three months). In some embodiments, the loading dose is 1500 mg subcutaneously every three days for a few weeks (e.g., two, three, or four weeks) to a few months (e.g., one, two, or three months)). In some embodiments, the loading dose is 1600 mg subcutaneously every three days for a few weeks (e.g., two, three, or four weeks) to a few months (e.g., one, two, or three months)). In some embodiments, the loading dose is 1800 mg subcutaneously every three days for a few weeks (e.g., two, three, or four weeks) to a few months (e.g., one, two, or three months)). In some embodiments, the loading dose ranges from about 500 mg to about 3,000 mg (and all intervals and integers therebetween) subcutaneously every three days for a few weeks (e.g., two, three, or four weeks) to a few months (e.g., one, two, or three months). The subcutaneous loading dose can be achieved in a single injection or in a few separate injections (e.g., two, three or four injections).

In one embodiment, the invention provides a method for treating a subject having HS comprising administering an anti-CD38 antibody, or antigen-binding portion thereof, or an anti-CD38 ADC, to the subject according to a multiple variable dose regimen, such that HS is treated, wherein the multiple variable dose regimen comprises administering a first loading dose, administering a second loading dose which is less than the first loading dose, and administering a treatment dose which is less than the second loading dose, wherein the treatment dose is administered to the subject weekly, for one to twelve weeks, and wherein each dose is separated between 7 to 28 days.

In one embodiment, the invention provides a method for treating a subject having HS comprising administering an anti-CD38 antagonist, e.g., an anti-CD38 antibody, or antigen-binding portion thereof, or an anti-CD38 ADC, to the subject according to a multiple variable dose regimen, such that HS is treated, wherein the multiple variable dose regimen comprises administering a first loading dose, administering a second loading dose which is less than the first loading dose, and administering a treatment dose which is less than the second loading dose, wherein the treatment dose is administered to the subject biweekly.

In one embodiment, the invention provides a method for decreasing the number of inflammatory lesions (AN count) in a subject having HS, said method comprising systemically administering an isolated human anti-CD38 antibody, or an antigen binding portion thereof, to the subject, such that the AN count is decreased. The decrease in AN count may be anything greater than 10%, e.g., the AN count may be reduced by at least a 50% reduction in the subject relative to baseline AN count. The subject may also exhibit other improvements in HS following treatment with an anti-CD38 antagonist, e.g., an anti-CD38 antibody, or antigen-binding portion thereof, or an anti-CD38 ADC. For example, the subject may have no increase in an abscess count and/or no increase in a draining fistula count following administration with an anti-CD38 antagonist, e.g., an anti-CD38 antibody, or antigen-binding portion thereof, or an anti-CD38 ADC.

In one embodiment, treatment of HS is achieved by administering an anti-CD38 antagonist, e.g., a human or humanized anti-CD38 antibody, or antigen-binding portion thereof, or an anti-CD38 ADC, to a subject having HS, wherein the human or humanized anti-CD38 antagonist, e.g., a human or humanized anti-CD38 antibody, or antigen-binding portion thereof, or an anti-CD38 ADC, is administered on a weekly or biweekly dosing regimen, or any combination thereof. Biweekly dosing regimens can be used to treat HS or symptoms associated with HS in which CD38 activity is detrimental. In one embodiment, biweekly dosing includes a dosing regimen wherein doses of an anti-CD38 antagonist, e.g., an anti-CD38 antibody, or antigen-binding portion thereof, or an anti-CD38 ADC, is administered to a subject every other week, e.g., beginning at week 1. In one embodiment, biweekly dosing includes a dosing regimen where doses of an anti-CD38 antagonist, e.g., an anti-CD38 antibody, or antigen-binding portion thereof, or an anti-CD38 ADC. are administered to a subject every other week consecutively for a given time period. e.g., 4 weeks, 8 weeks, 16, weeks, 24 weeks, 26 weeks, 32 weeks, 36 weeks, 42 weeks, 48 weeks, 52 weeks, 56 weeks or more, etc. Biweekly or weekly dosing is preferably administered parenterally, including intravenously, and/or subcutaneously. In one embodiment, the anti-CD38 antagonist, e.g., a human or humanized anti-CD38 antibody, or antigen-binding portion thereof, or an anti-CD38 ADC, is administered in a dose of about 20 mg to about 2,000 mg intravenously or subcutaneously as a total daily dose for each weekly or biweekly dosing. In one embodiment, the human CD38 antibody, or an antigen-binding portion thereof, is adalimumab. Additional examples of dosing regimens within the scope of the invention are provided herein in the In one embodiment, treatment of HS is achieved using multiple variable dosing methods of treatment. For example, a loading dose ranging from about 80 to about 3,000 mg of an anti-CD38 antagonist, e.g., a human or humanized anti-CD38 antibody, or antigen-binding portion thereof, or an anti-CD38 ADC, may first be administered to a subject having HS, followed by a maintenance or treatment dose ranging from about 100 to about 2,000 mg.

In one embodiment, the invention provides a method of treating HS in a subject comprising administering a loading dose(s) of an anti-CD38 antagonist, e.g., a human or humanized anti-CD38 antibody, or antigen-binding portion thereof, or an anti-CD38 ADC, to the subject at week 0, optionally another loading dose at week 2. In one embodiment, the loading dose(s) is given in its entirety on one day or is divided over multiple days (e.g., divided over two days). In one embodiment, the loading dose(s) is administered subcutaneously. Following administration of the loading dose(s), one or more maintenance or treatment dose(s) of the anti-CD38 antagonist, e.g., a human or humanized anti-CD38 antibody, or antigen-binding portion thereof, or an anti-CD38 ADC, may be administered to the subject, wherein the maintenance or treatment dose is about half or ¼ of the dose amount of the loading dose(s). In one embodiment, the maintenance or treatment dose is administered to the subject about one or two weeks after the last loading dose(s). In one embodiment, the maintenance or treatment dose is administered subcutaneously. Subsequent doses may be administered following the same or different maintenance or treatment dosing regimen.

In one embodiment, the first loading dose is about 140 to about 3,000 mg (and all intervals and integers therebetween). Numbers intermediate to the stated range are also included in the invention, e.g., 138-175 mg, 150-170 mg, and 155-165 mg. In one embodiment, the first loading dose ranges from about 100 mg to about 2,000 mg (and all intervals and integers therebetween).

In one embodiment, the second loading dose is about 40 to about 3,000 mg (and all intervals and integers therebetween). Numbers intermediate to the stated range are also included in the invention, e.g., 38-90 mg, 75-85 mg, and 65-95 mg. In one embodiment, the second loading dose ranges from about 80 mg to about 2,000 mg (and all intervals and integers therebetween).

In one embodiment, the treatment dose is about 20-50 mg. Numbers intermediate to the stated range are also included in the invention, e.g., 25-38 mg. In one embodiment, the treatment dose is about 40 mg.

In another embodiment, the loading dose(s) of the human anti-CD38 antibody, or antigen-binding fragment thereof, comprises about 80 mg, and may be given at week 0, followed by at least one maintenance dose of the anti-CD38 antagonist, e.g., a human or humanized anti-CD38 antibody, or antigen-binding portion thereof, or an anti-CD38 ADC, comprising about 40-2,000 mg, administered on a biweekly dosing regimen, optionally from week 1. Alternatively, in another embodiment, the loading dose(s) of the anti-CD38 antagonist, e.g., a human or humanized anti-CD38 antibody, or antigen-binding portion thereof, or an anti-CD38 ADC, comprises a first dose of about 50 mg to about 2,000 mg administered on week 0, and a second loading dose ranging from about 80 mg to about 2,000 mg administered on week 2, followed by at least one maintenance dose of the anti-CD38 antagonist, e.g., a human or humanized anti-CD38 antibody, or antigen-binding portion thereof, or an anti-CD38 ADC, comprising about 40 to about 2,000 mg, administered weekly thereafter. In one embodiment, the maintenance dose is administered to the subject starting at about week 4 (wherein week 0 is the initial loading dose).

In one embodiment, the subject is first selected for having HS and is then administered a anti-CD38 antagonist, e.g., a human or humanized anti-CD38 antibody, or antigen-binding portion thereof, or an anti-CD38 ADC, in accordance with the methods described herein.

Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. Such dosage unit forms may be a tablet or pill with a pre-determined amount of therapeutic agents, or a vial with therapeutic agents to be reconstituted by a solution to produce a drug product of a pre-determined final concentration. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

Dosage regimens described herein may be adjusted (e.g., in individual patients) to provide the optimum desired response, e.g., maintaining remission of HS, in consideration of the teachings herein.

It is to be noted that dosage values can vary with the type and severity of HS. It is to be further understood that for any particular subject, specific dosage regimens may be adjusted over time according to the teachings of the specification and the individual need and the professional judgment of the person administering or supervising the administration of the compositions, and that dosage amounts and ranges set forth herein are exemplary only and are not intended to limit the scope or practice of the claimed invention.

### HS Patient Populations

The present disclosure provides uses and methods for treating certain subpopulations of HS patients with an anti-CD38 antagonist, e.g., a human or humanized anti-CD38 antibody, or antigen-binding portion thereof, or an anti-CD38 ADC.

In one embodiment, the subject has HS lesions in at least two distinct anatomic areas prior to treatment.

In one embodiment, the subject had an inadequate response to or was intolerant to oral antibiotics for treatment of their HS.

In one embodiment, the subject has an AN count of greater than or equal to 3 at baseline, a female, a subject who is over 40 years old, a subject who is a smoker, or any combination thereof.

In one embodiment, the invention provides a method of treating moderate to severe HS in a subject comprising administering to the subject a CD38 inhibitor, e.g., an anti-CD38 antibody, or antigen-binding fragment thereof, such that mild, and moderate to severe HS is treated. Subjects having mild, and moderate to severe HS may be administered an anti-CD38 antagonist, e.g., a human or humanized anti-CD38 antibody, or antigen-binding portion thereof, or an anti-CD38 ADC, such that mild, and moderate to severe HS is treated and advancement of the disease is prevented. The invention also provides use of an anti-CD38 antagonist, e.g., a human or humanized anti-CD38 antibody, or antigen-binding portion thereof, or an anti-CD38 ADC, in the manufacture of a medicament for the treatment of mild, and moderate to severe HS in a subject who has mild, and moderate to severe HS. In one embodiment, a patient having mild, and moderate to severe HS is defined as a patient having a HS-PGA score no less than 2. In one embodiment, such patients have been unresponsive or intolerant to oral antibiotics for treatment for their HS. In one embodiment, such patients have had a diagnosis of mild, and moderate to severe HS for at least 6 months prior to Baseline HS-PGA measurement, and involve at least two distinct anatomic areas (e.g. left and right axilla; or left axilla and left inguinal-crural fold).

### Articles of Manufacture and Kits

The present disclosure also provides articles of manufacture comprising an agent that specifically binds to human CD38. In some embodiments, an article of manufacture of the present disclosure comprises an anti-CD38 antagonist, e.g., a human or humanized anti-CD38 antibody, or antigen-binding portion thereof, or an anti-CD38 ADC, described herein. In certain embodiments, the article of manufacture comprises Daratumumab®. The present disclosure further provides methods for manufacturing said articles.

The present disclosure also provides kits comprising an anti-CD38 antagonist that specifically binds to human CD38, or inhibits its expression or activity, as well as instructions for the use of the agent or combination of active agents, wherein one of the active agents is an anti-CD38 antagonist, e.g., a human or humanized anti-CD38 antibody, or antigen-binding portion thereof, or an anti-CD38 ADC. In some embodiments, a kit of the present disclosure comprises an anti-CD38 antibody or an antigen-binding fragment thereof, or an anti-CD38 ADC, or combinations thereof, described herein. In certain embodiments, the kit comprises Daratumumab® or SARCLISA® (isatuximab-irfc, anti-CD38 antibody); or Daratumumab® or SARCLISA® (isatuximab-irfc, anti-CD38 antibody) conjugated to a cytotoxic agent, optionally with one or more secondary agents, including, but not limited to, an antibiotic, an anti-inflammatory, an immunosuppressant agent, a chemotherapeutic agent, or combinations thereof.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification. including definitions, will control.

Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein.

Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers.

All publications and other references mentioned herein are incorporated by reference in their entirety. Although a number of documents are cited herein, this citation does not constitute an admission that any of these documents forms part of the common general knowledge in the art.

### EXAMPLES

The Examples in this specification are not intended to, and should not be used to, limit the invention; they are provided only to illustrate the invention.

### Example 1. Disease Biomarkers and Targets for HS Therapeutic Treatment

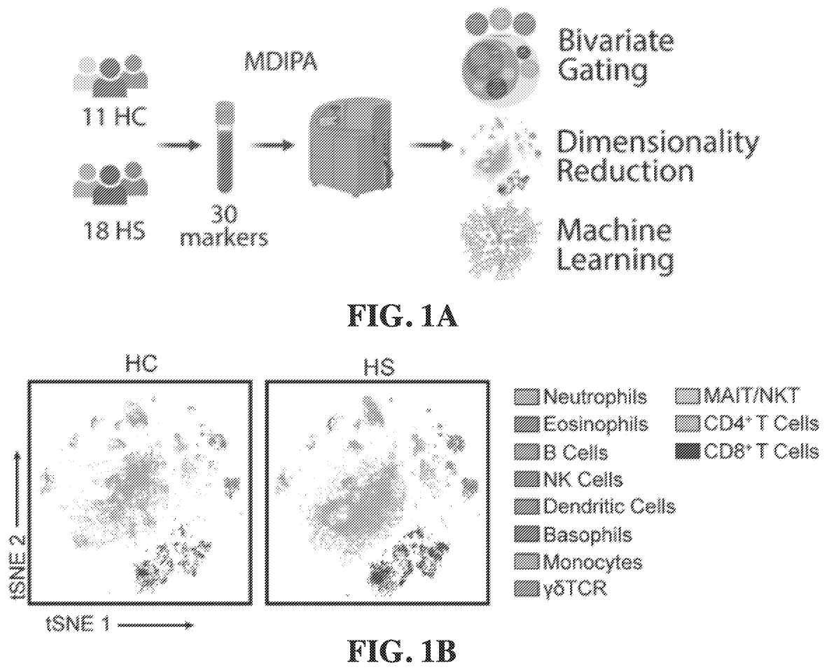

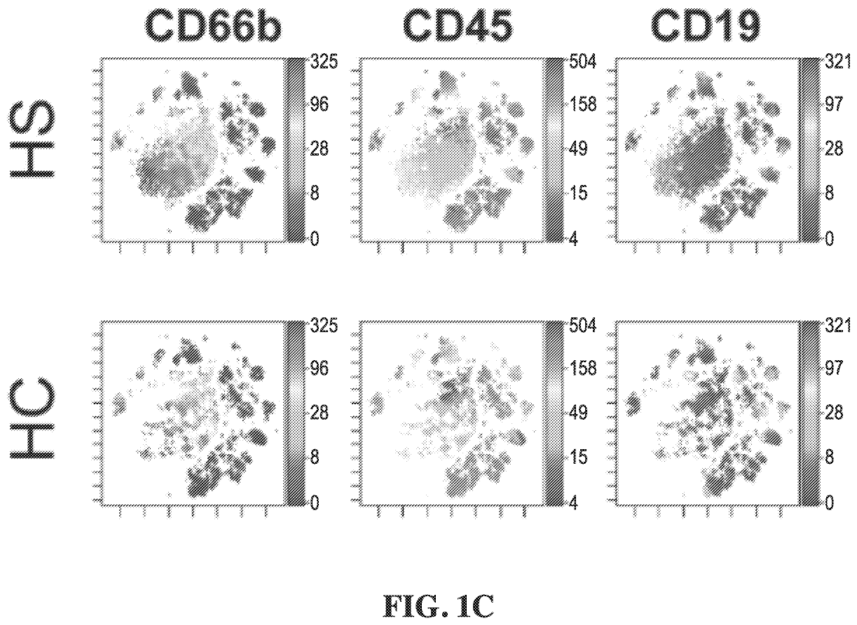

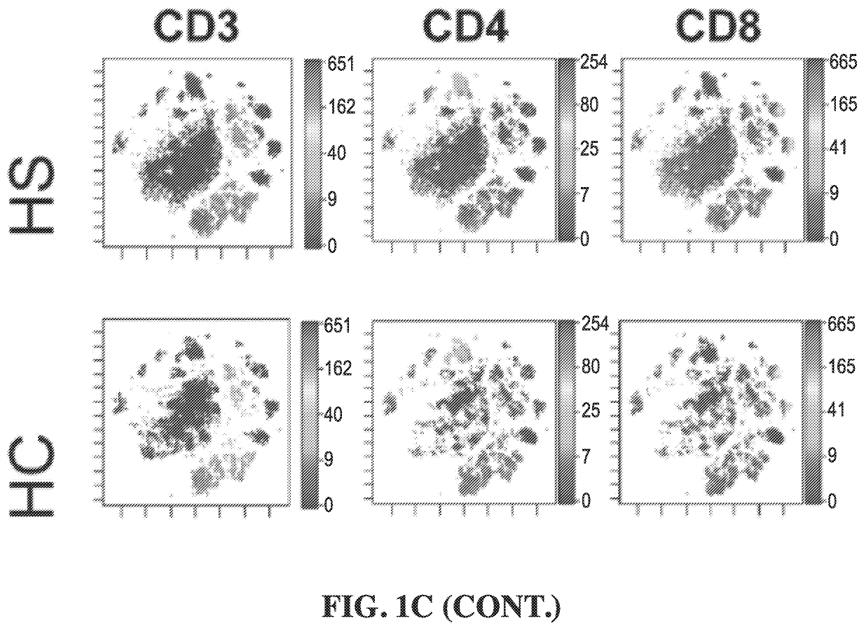

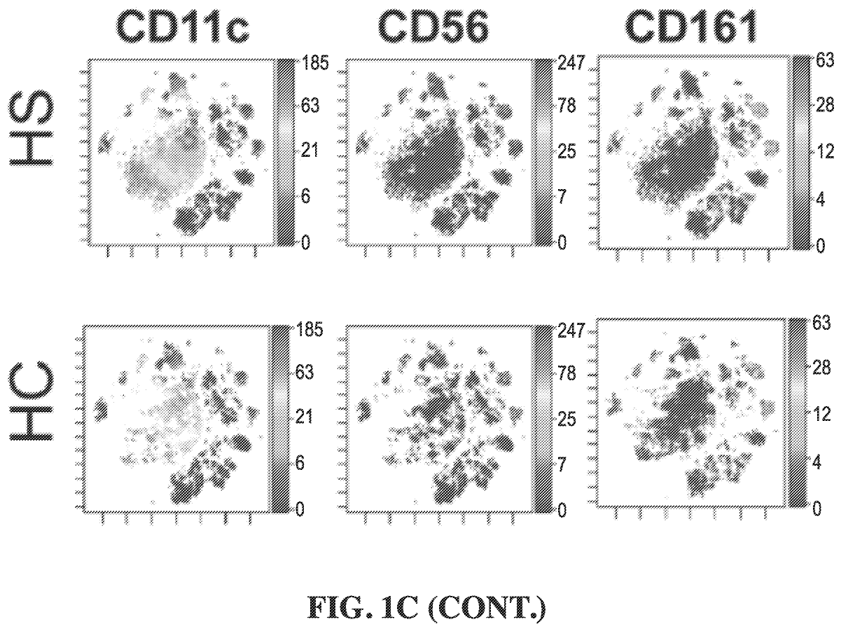

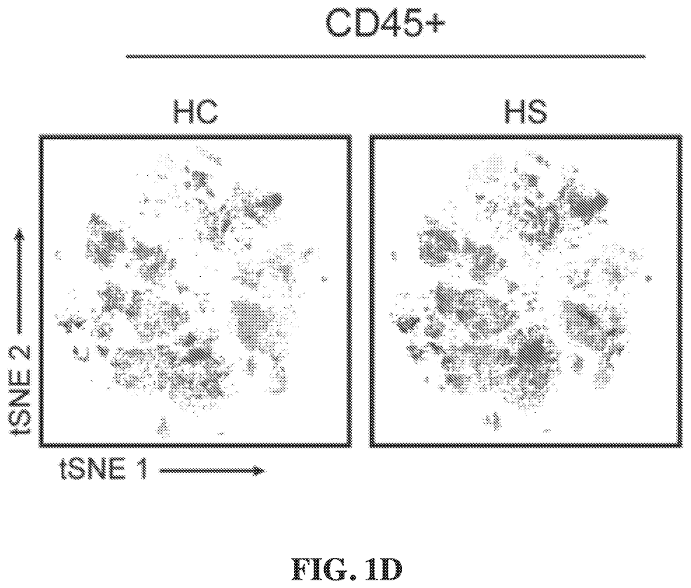

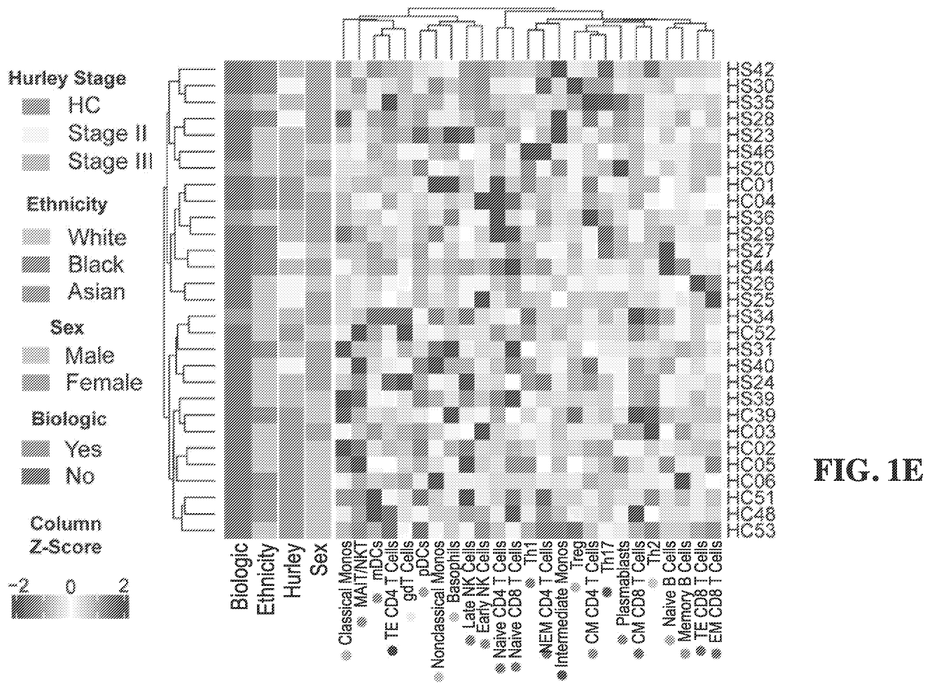

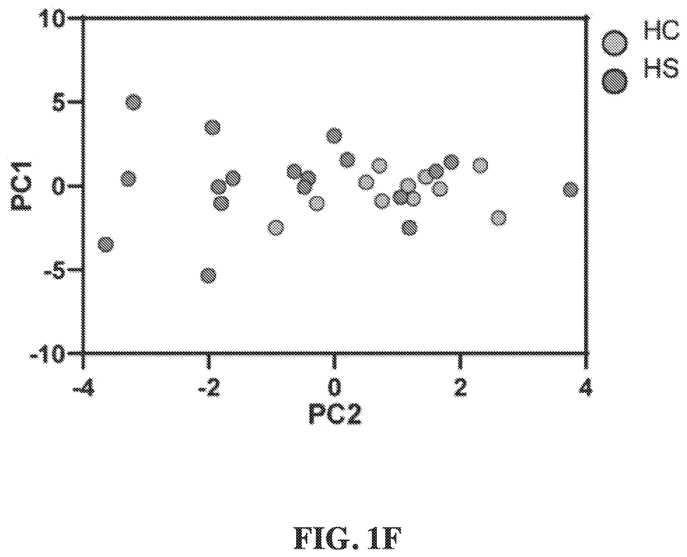

To define the dysregulation of the immunome in hidradenitis suppurativa (HS) patients, the peripheral and localized immunome changes in HS patients were compared to healthy controls (HC). The peripheral immunome was characterized by mass cytometry (CyTOF) with bivariate gating and machine-learning algorithms applied to the high-dimension single cell data to identify dysregulated subpopulations of cells in HS (FIG. 1). A bulk RNA-sequencing Meta-Analysis of gene expression changes in HS skin was performed using crowd-sourced public RNA sequencing data from HS and HC skin samples. Changes in immune cell population in HS skin were identified using bivariate flow cytometry, imaging mass cytometry, and immunohistochemistry staining. Altogether, the data from studies in Example 1 identified disease biomarkers and CD38 as a target for HS therapeutic treatment.

### Methods

The schematic in FIG. 1A shows the CyTOF assay procedure on fresh whole blood from 18 patients with HS (6-Hurley Stage II; 12-Hurley Stage III) and 11 healthy control patients (HC) using Maxpar Direct Immunoprofiling Assay (MDIPA).

### Human Subjects

Blood samples were obtained from individuals who provided written informed consent, and samples were deidentified prior to processing under guidelines approved by the Henry Ford Health Institutional Review Board. Patients with hidradenitis suppurativa (HS) were enrolled in Detroit, Michigan during regularly scheduled medical appointments. Patients were staged and diagnosed by an expert dermatologist who focuses on treating patients with HS at the HFHS HS specialty clinic. All patients were staged at Hurley stage II or stage III, and demographic information is provided in Table 1 below. Healthy control (HC) blood was collected at Henry Ford Health from volunteers with no previous history of skin or autoimmune disease. All blood samples were collected in cell preparation tubes (CPT tubes, BD Biosciences, San Jose, CA) containing sodium heparin and were processed for mass cytometry within 4 h of collection.

TABLE 1
Demographic characteristics of patients
with hidradenitis suppurativa

| Characteristic | HS: n (%) | | HC: n (%) |
|---|---|---|---|
| Ethnicity | | | |
| White | 11 | (61%) | 5 (45%) |
| Black | 7 | (39%) | 4 (37%) |
| Other | 0 | (0%) | 2 (18%) |
| Sex | | | |
| Male | 14 | (78%) | 9 (82%) |
| Female | 4 | (22%) | 2 (18%) |
| Hurley Stage | | | N/A |
| Stage II | 6 | (33.3%) | |
| Stage III | 12 | (66.7%) | |

### Mass Cytometry Time-of-Flight (CyTOF)

Fresh blood was processed and stained using the Maxpar Direct Immunoprofiling Assay (MDIPA) (Fluidigm or The Longwood Medical Area CyTOF core, Boston, MA). Following processing and staining, samples were stored at −80° C. The frozen stained blood samples were placed at room temperature (RT) for 30-50 min until fully thawed and then incubated with 1× Thaw-Lyse buffer (Smart Tube Inc.) at RT for 10 min. The lysis steps were repeated up to 3 times until the pellet turned white evidencing complete lysis of red blood cells. The cells were then incubated with Maxpar Fix and Perm Buffer (Fluidigm) containing 125 nM Cell-ID Intercalator-Ir solution (Fluidigm) at 4° C. overnight or up to 48 h before sample acquisition. Groups of samples (4-8/day) were assessed by Helios mass cytometry (Fluidigm) in 4 independent experiments using a flow rate of 45 μl/min in the presence of EQ Calibration beads (Fluidigm) for normalization. An average of 400,000 f 50,000 cells (mean SEM) from each sample were acquired and analyzed by Helios. Gating was performed on the Cytobank platform (Cytobank, Inc. Santa Clara, CA) and FlowJo 10.5.3 (BD Biosciences).

### CyTOF Data Processing

FCS files generated by CyTOF were normalized and concatenated, if necessary, using CyTOF Software version 6.7. All CyTOF processed files were also uploaded to the Cytobank, a cloud-based analysis platform. Beads, debris, doublets, and dead cells were manually removed by sequential gating per the Approach to Bivanate Analysis of Data Acquired Using the Maxpar Direct Immune Profiling Assay (Fluidigm, Technical Note). The total live cells or CD45+CD66b− live singlets were selected for viSNE analysis or gated manually with multiple cell lineage markers to define immune populations.

Flow Cytometry from PBMCs

Samples of peripheral blood mononuclear cells (PBMC) were thawed rapidly and transferred with 2 ml R1640 medium containing 10% FBS and 50 U/ml benzonase to a fresh 15 ml tube. Then, 5 ml of R1640 medium containing 10% FBS was added, and samples were pelleted by centrifugation for 10 minutes at 300 rpm. The medium was aspirated, and the pellets were resuspended in 2 ml of RPMI 1640 containing 10% FBS and transferred to a culture plate to be placed at 37° C. 5% CO2 for 2 hours to rest. Samples were then centrifuged, and pellets were resuspended in staining buffer (2% FBS, 1×PBS). Samples were incubated with Human TruStain FcX block (Biolegend) for 15 minutes and then were stained with cell surface antibodies for 30 minutes at RT. The following conjugated antibodies were used: CD3 (UCHT1), CD4 (OKT4), CD8 (SK1), CCR4 (LZ91H4), and CCR6 (G034E3), all purchased from Biolegend. Following staining, samples were washed twice with PBS. For intracellular cytokine staining, cells were fixed and permeabilized with IC fixation buffer (Invitrogen) and Permeabilization buffer (Invitrogen) using manufacture's specifications. Samples were then stained with intracellular markers: IL-17 (BL168), TNFα (Mab11), and IFNγ (4S.B3). Flow cytometry assay was performed using a BD FACSCelesta instrument and data were analyzed with the FlowJo V10.2 software.

### Bulk RNA-Sequencing Meta-Analysis

Fastq files were downloaded from the Gene Expression Omnibus for accession numbers GSE151243, GSE154773, and GSE155176, resulting in 130 unique samples. Raw sequence reads were adapter trimmed and quality control assessed. he HISAT2 alignment program was used with default settings to align the paired end reads to Genome Reference Consortium Human Build 38 (CRCh38). Only uniquely mapped reads were used for subsequent analyses. Principal component analysis was used to identify potential batch effects arising from the different data sources. DESeq2 was used to normalize counts and identify differentially expressed genes between sample types.

### Immunohistochemistry

FFPE (formalin-fixed paraffin-embedded) tissues were cut to 4 μm sections, placed on slides and deparaffinized. Slides were dried at 60° C. and then incubated in FLEX Target Retrieval Solution (TRS) at an optimized pH (high or low). Staining was performed for CD38 (Cell Signaling E7Z8C; 1:100, TRS-high pH), CD16 (Cell Signaling D1N9L; 1:500, TRS-low pH), CD56 (Agilent 123C3; TRS-high pH), and CD14 (Cell Signaling D7A22T; 1:250, TRS-low pH) on a Dako Autostainer Link. Slides were washed, dehydrated, mounted, and scanned on a Leica Biosystem Aperio CS2 light microscope.

### Immunohistochemistry Scoring

Three independent raters scored immunohistochemistry staining from 0 to 3, where 0 was a complete absence of staining and 3 was equivalent to a positive control. Each data point represents the average of the 3 raters' scores.

### Tissue Preparation and Staining for Imaging Mass Cytometry

All tissue samples had previously been formalin-fixed and paraffin embedded for routine diagnostic pathology at Henry Ford Hospital within the past 5 years. Representative regions of interest (ROI) were selected from lesional and perilesional sections using hematoxylin and eosin staining. Two tunnel regions were captured from lesional sections. Tissue sections were cut at 4 μm. Slides were baked for 1 hour at 60° C. and then dewaxed in xylene ×3 for 3 minutes each. Tissue sections were rehydrated in ethanol 100%×3 for 3 minutes each, 95% ethanol ×2 for 1 minute each, and water ×1 for 1 minute. Slides were fixed in 10% neutral buffered formalin for 30 minutes to help prevent tissue movement Antigen retrieval was performed using Target Retreival Solution, high pH (Dako) for 30 min at 95° C. in a Decloaking Chamber (Biocare Medical). Slides were allowed to cool and placed in wash buffer. Blocking was performed with Background Sniper (Biocare Medical) at RT for 15 minutes. Samples were incubated overnight in a humidity chamber at 4° C. in the primary antibody master mix diluted in TBS with 1% BSA (Sigma Aldrich). Slides were washed twice in 0.1% TWEEN20 (Sigma Aldrich) in TBS, twice with TBS, then stained with Ir-Intercalator-Ir (Fluidigm) in PBS for 30 minutes at RT. Slides were then washed in water for 5 minutes and allowed to air dry. All samples were simultaneously processed and stained.

Imaging mass cytometry Data Acquisition

Images were acquired using a Hyperion Imaging System (Fluidigm). The largest square area from each region of interest was laser-ablated at 200 Hz and subsequent images were rendered using MCD Viewer software (Fluidigm). A total of 16 region of interests from 3 lesional and 3 perilesional sections from patients with Hurley Stage III HS were assayed.

### Imaging Mass Cytometry Data Analysis

Using the Steinbock Docker45 container on Linux, the mass cytometry images and regions of interest were extracted and annotated from the raw mcd files. Cell segmentation was performed using DeepCell (Mesmer)61 contained in Steinbock, which utilizes a pretrained deep learning model to distinguish between cell and non-cell regions using spatially resolved protein expression from cell nuclei (DNA1 and DNA2) and cytoplasm/membrane markers (ICSK2, CD45, Keratin, K10, K14, aSMA, CD1a, CD3, CD16, CD68, CD163). The cell segmentation process results in a mask, showing the shape and location of the identified cells, sharing the same xy coordinates as the stained imaging mass cytometry slides. For objects identified in the masks, mean pixel intensities for the markers used in the staining panel were extracted.

The resultant summary features were read into R4.2.0 using the imcRtools package 45. Initial unsupervised clustering was performed using Rphenograph, taking into consideration the expression of cell-type specific markers. Aberrant clusters, which contained a very small percentage of cells with variable expression of non-biologically relevant markers, were removed from downstream analysis to eliminate staining artifacts. After reclustering the remaining cells, phenotypically similar cell clusters were merged into larger metaclusters to define biologically relevant populations. Spatial patches of cell groupings were identified using the imcRtools “patchDetection” function with endothelial cell and mono-mac populations as a reference point.

### Statistical Analysis

Dimensionality reduction, presentation, and statistical analyses for CyTOF and IMC data were carried out using Prism 9 software (GraphPad) or R (v4.2.1). Data was plotted and graphed as mean f standard deviation of the mean. Tests for normality were conducted and the appropriate parametric or nonparametric tests were utilized. CITRUS analysis was conducted on the Cytobank webserver utilizing its own built-in statistics. Cluster size, event sampling, and cross validation folds were adjusted to minimize the model-error rate. Significant clusters were designated by a false-discovery rate (FDR)<0.01. Deriving a CD38 associated gene-signature was accomplished by finding all genes with significant correlation to CD38, FDR <0.05 and r>0.5.

### Results

### 1.1 Characterization of the Peripheral Immunome by CyTOF

A T-distributed stochastic neighbor embedding (t-SNE) dimensionality reduction defined 11 major clusters of immune cells in whole blood, which were corroborated by lineage marker expression (FIG. 1B-C).

### Bivariate Gating CyTOF

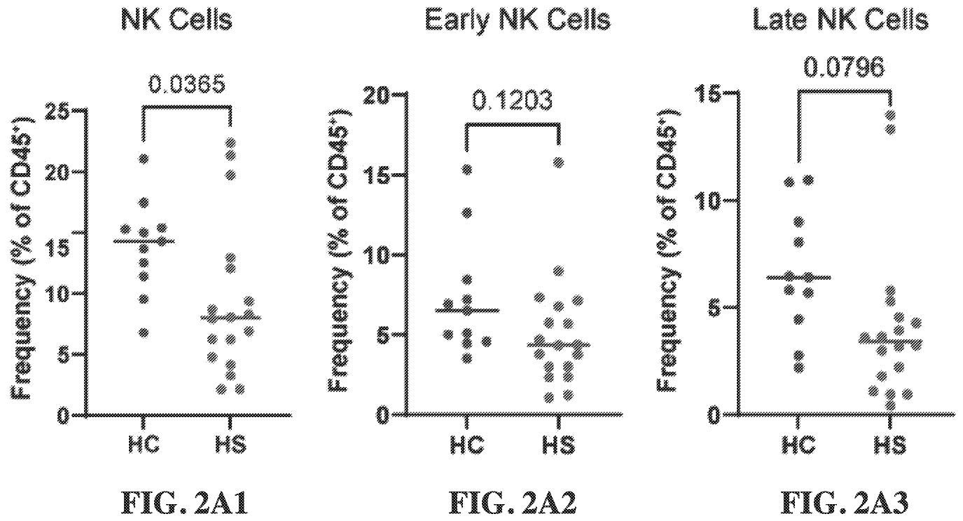

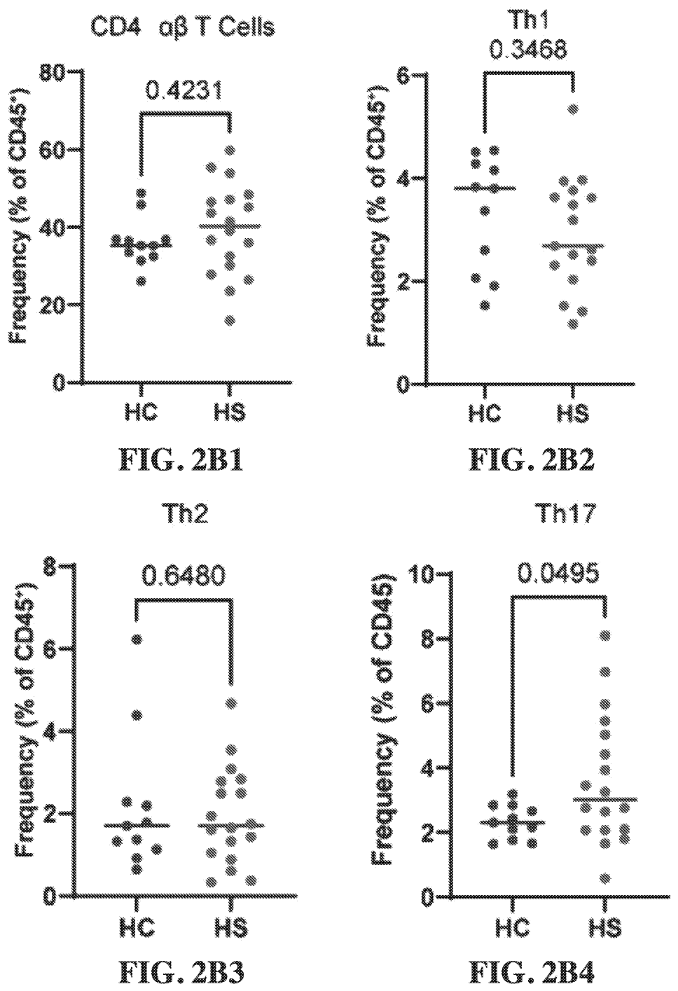

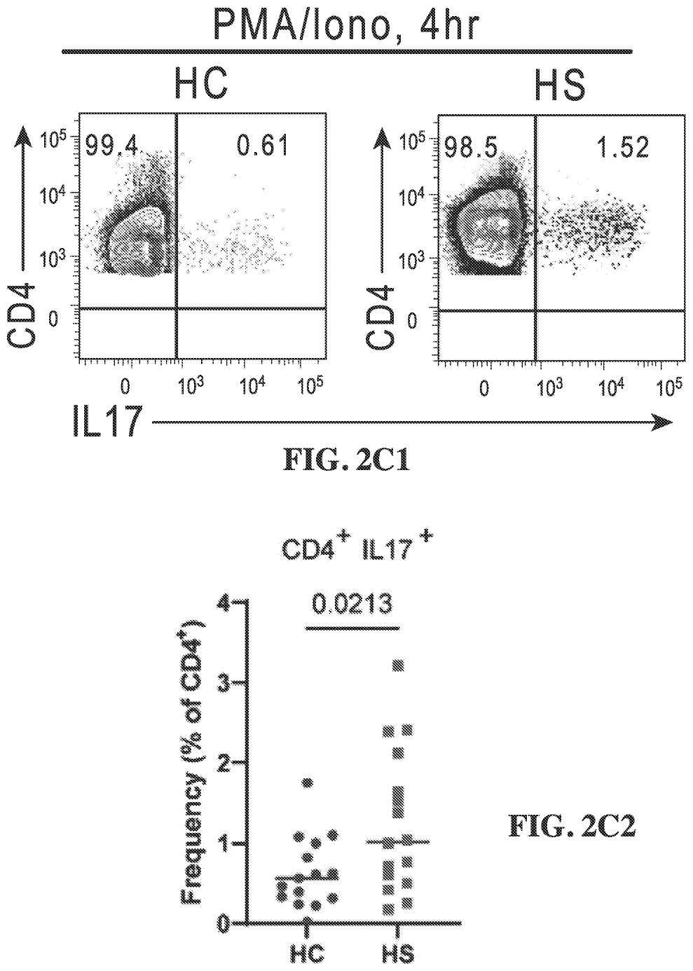

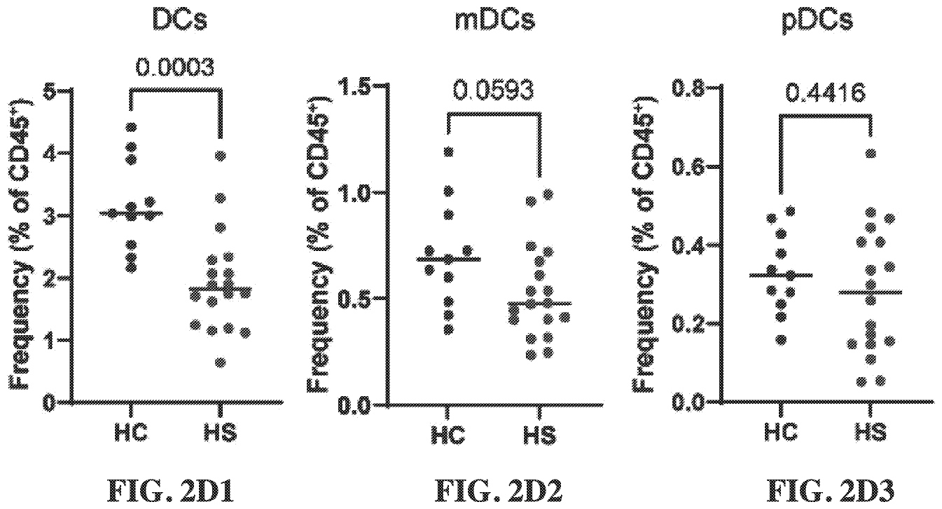

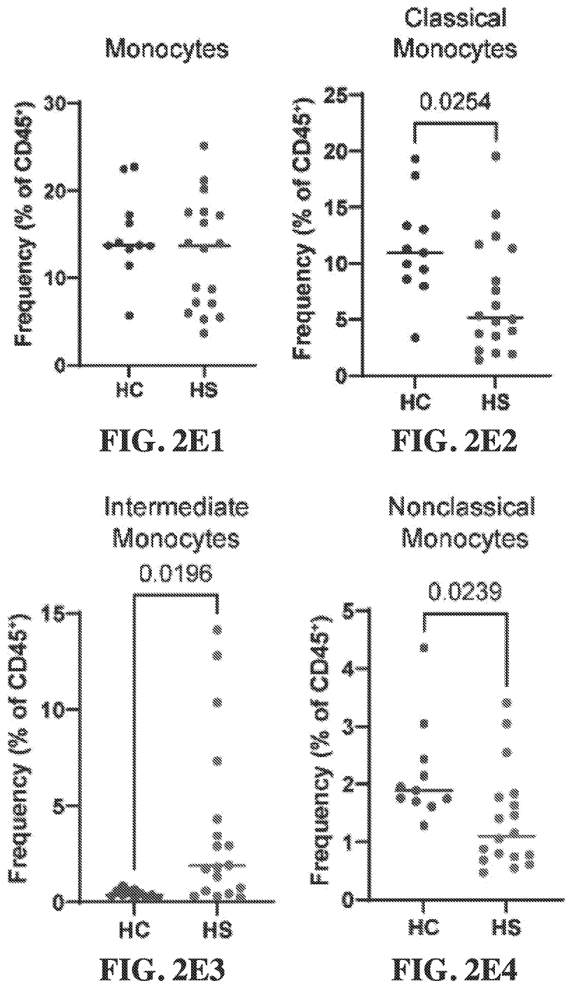

Using bivariate gating, 25 CD45+ cell subsets were identified (FIG. 1D-IE). Hierarchical clustering of the frequencies of these subsets showed heterogeneity between the immunomes of patients with HS unexplained by use of biologic drugs, sex, ethnicity, or Hurley Stage (FIG. 1E). The Hurley staging system categorizes patients into 3 groups based on severity of skin pathology. PCA analysis of CD45+ immune subsets showed a more pronounced separation between HS and HC cell populations (FIG. 1F). HS patients had a significant reduction in the frequency of circulating NK cells, with a decreasing trend in both early and late NK cell subsets in HS (FIG. 2A1-3). No differences were observed in the frequency of total CD4+αβT cells between HS and HC, but the frequency of the Th17 subset was elevated in HS (FIG. 2B1-4).

The functional capacity of Th17 cells was tested between HS and HC by stimulating peripheral blood mononuclear cells (PBMCs) (from an independent cohort of patients with HS) with phorbol myristate acetate and ionomycin (PMA/Iono) for 4 hours. Patients with HS had a significantly greater frequency of CD4+IL-17+ cells, indicating that circulating Th17 cells in patients with HS have an increased intrinsic ability to produce IL-17 (FIG. 2C1-2). These results show that patients with HS have increased frequency and function of circulating h17 cells. The frequency of monocyte-derived DCs was lower in HS patients compared to HC (p=0.0593), but HC patients showed similar frequency of plasmacytoid DCs compared to HCs (p=0.4416) (FIG. 2D1-3). No difference in total monocyte frequency was found between HS and HC (FIG. 2E1-4). However, CD14 and CD16 expression analysis showed significantly reduced numbers of classical monocytes and non-classical monocytes and increased level of intermediate monocytes in HS patients compared to HC. The data demonstrates immune cell dysregulation in the peripheral immune system of HS patients.

### Machine Learning Algorithms

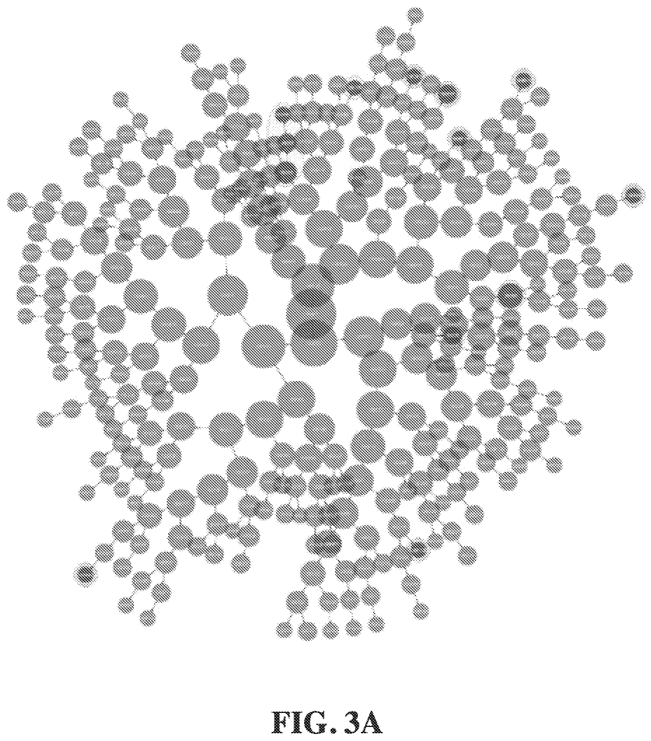

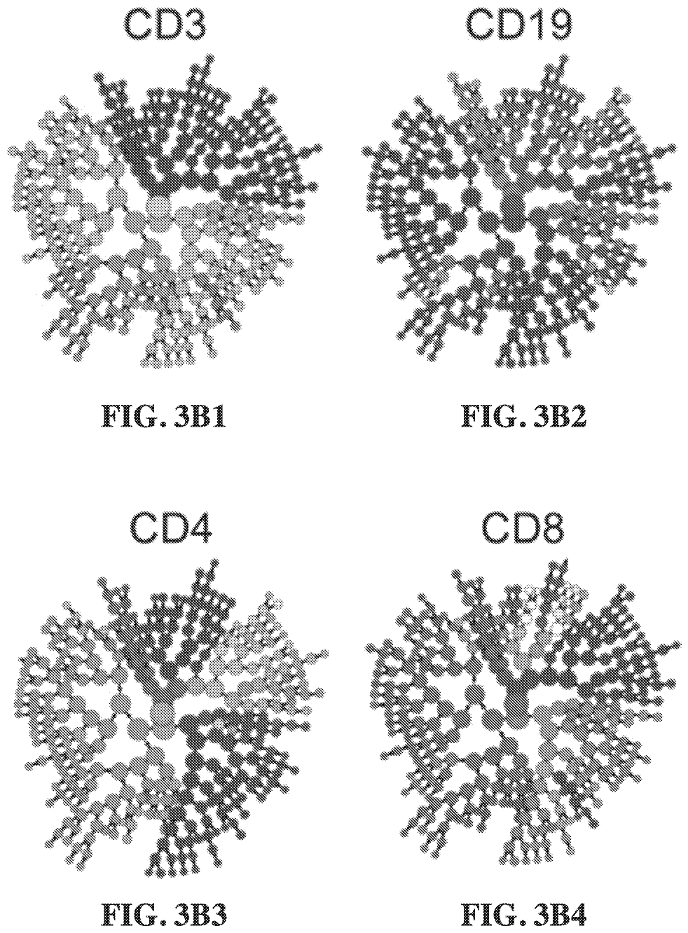

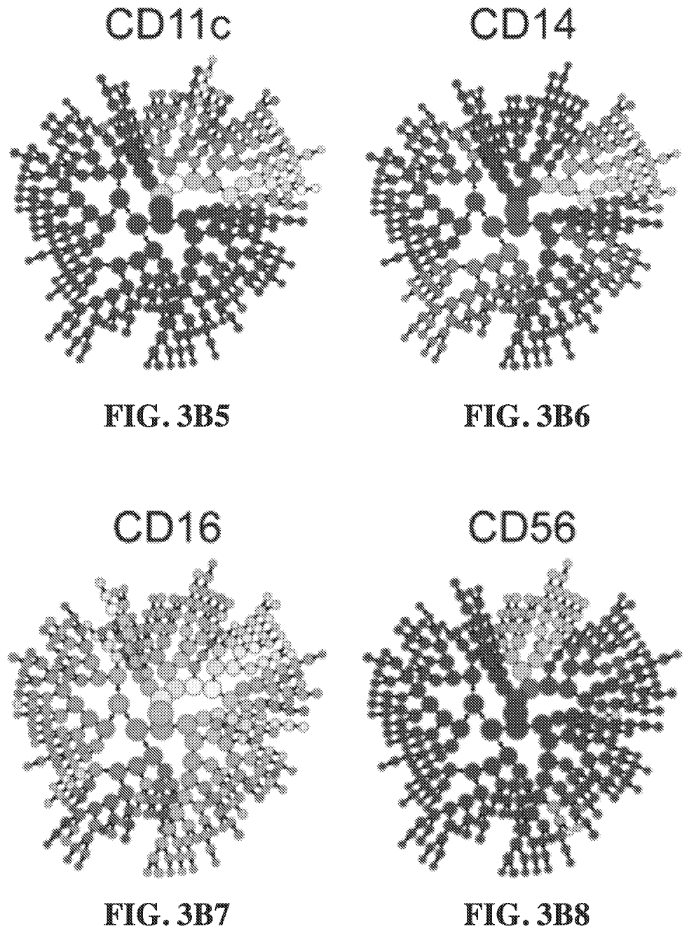

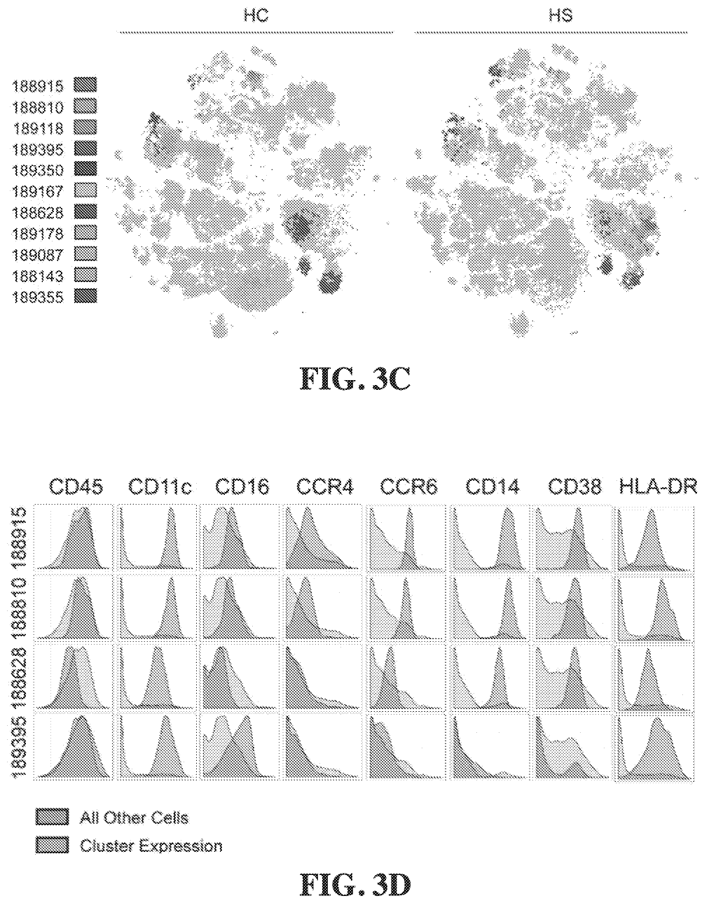

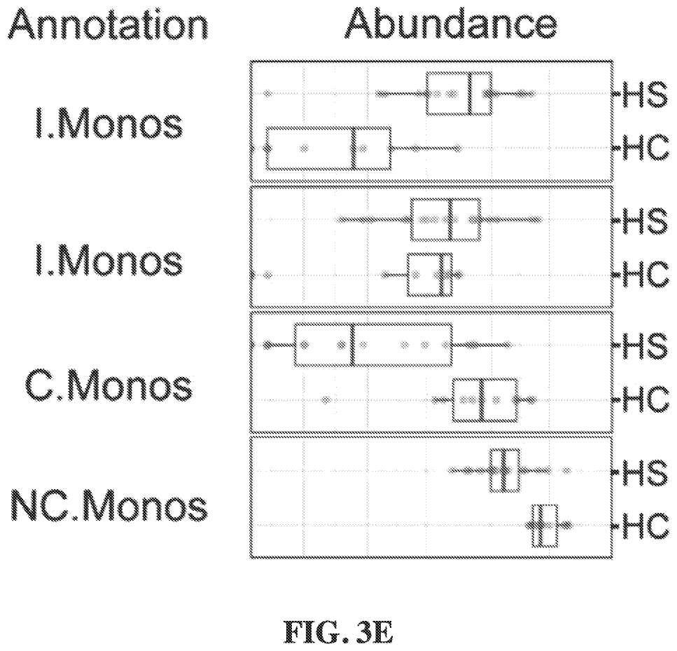

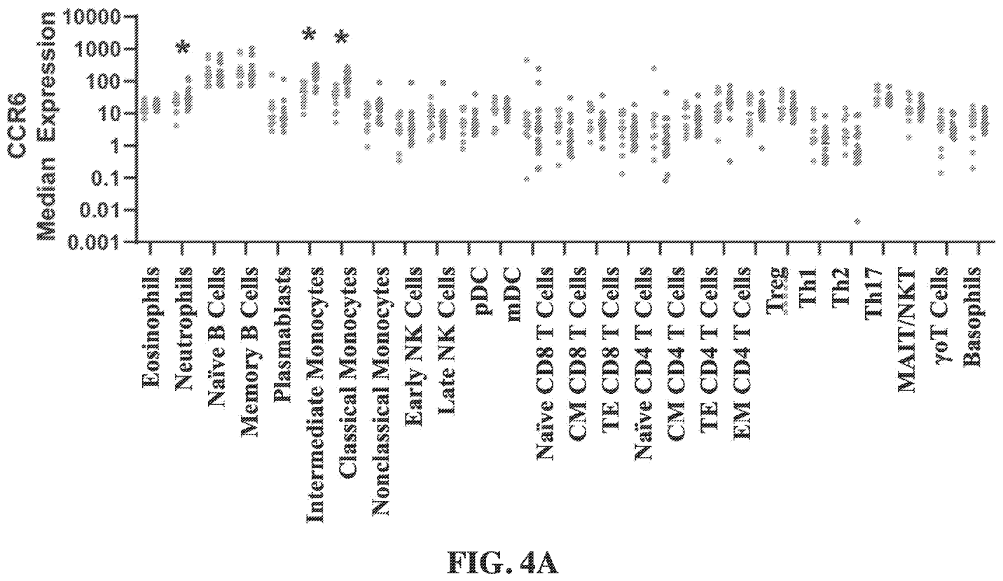

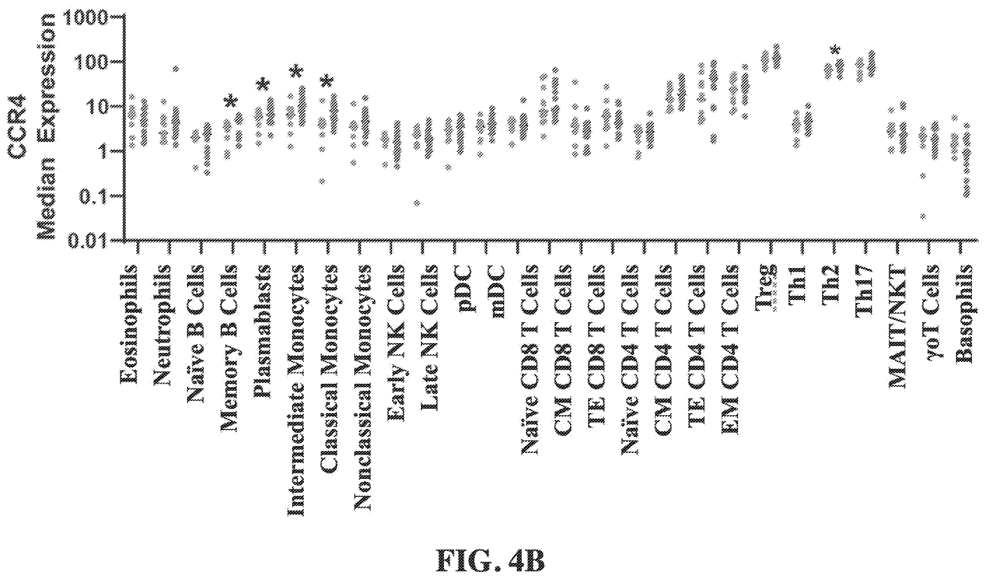

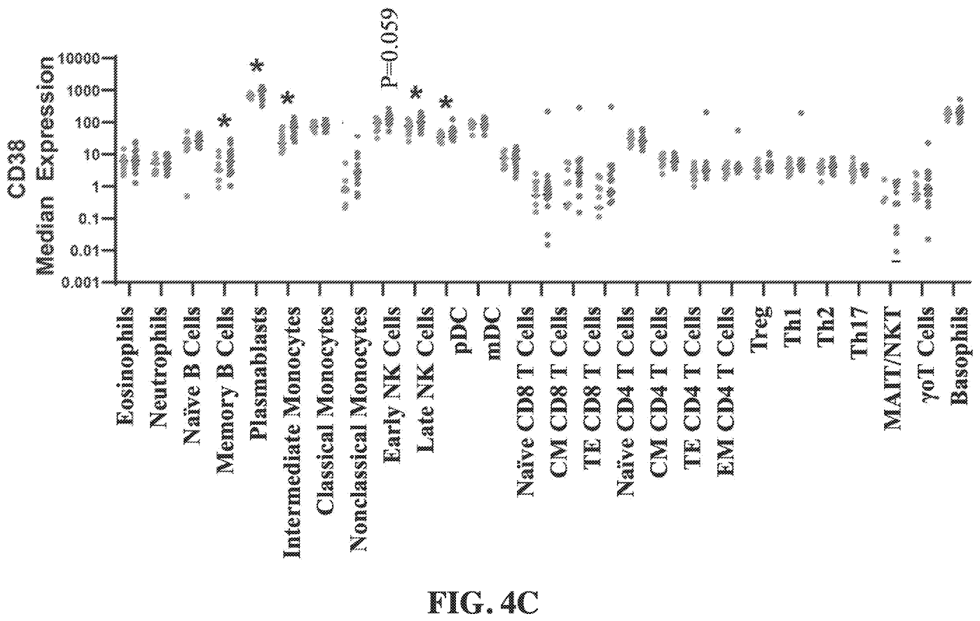

Machine-learning algorithms applied to high-dimensional single-cell data can perform unbiased clustering and statistical comparison of clusters between conditions to identify previously undescribed cell subpopulations. An L1-penalized regression implementation of the cluster identification, characterization, and regression (CITRUS) analysis confirmed findings from the bivariate gating analysis. Unbiased clustering of CD45+ cells found 11 differentially abundant clusters that corresponded to 9 subpopulations (FIGS. 3A & B1-8). The cells were mapped back into T-distributed stochastic neighbor embedded space and the expression pattern of lineage markers analyzed to determine cell identity (FIG. 3C-D). The CITRUS results confirmed that intermediate monocytes were more abundant, but non-classical monocytes, classical monocytes, and NK cells were decreased in patients with HS (FIG. 3D-E). The high level of CD38 expression in intermediate monocytes in CITRUS clusters, which do not express CD38 appreciably, indicates that intermediate monocytes are activated in HS patients (FIG. 3D). Intermediate and classical monocytes from HS patients also expressed a higher level of CCR4 and CCR6, which are receptors for chemokines released from inflamed skin (FIG. 3D, 4A-B). As shown in FIGS. 4A and 4B, CCR6 expression is elevated in neutrophils, and intermediate and classical monocytes, whereas CCR4 expression is elevated in memory B cells, plasmablasts, intermediate and classical monocytes, and Th2 cells from HS patients. CD38 expression is increased in memory B cells, plasmablasts, intermediate monocytes, late NK cells, and plasmacytoid DCs (pDCs) in blood from HS patients (FIG. 4C).

### CD38 Expression Gating

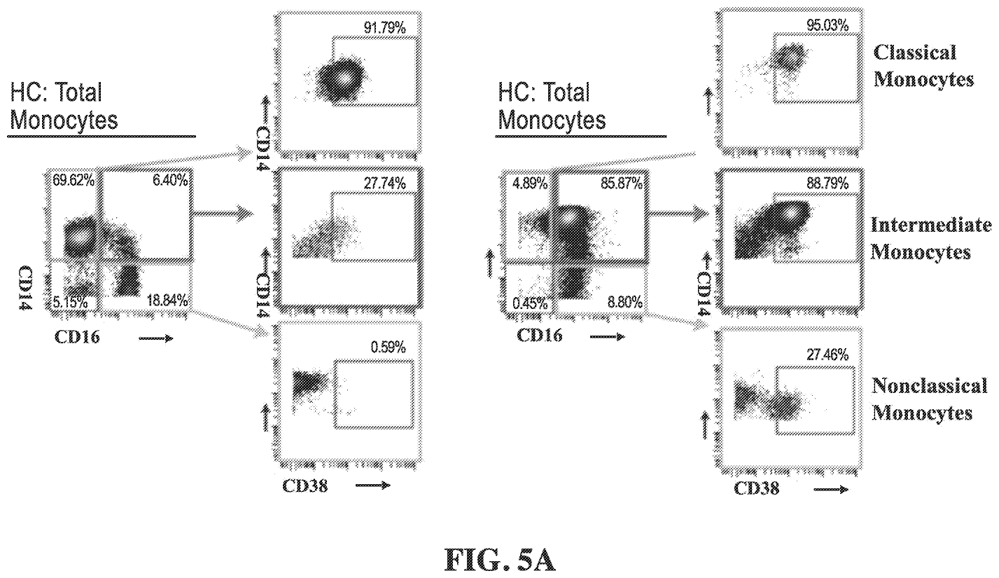

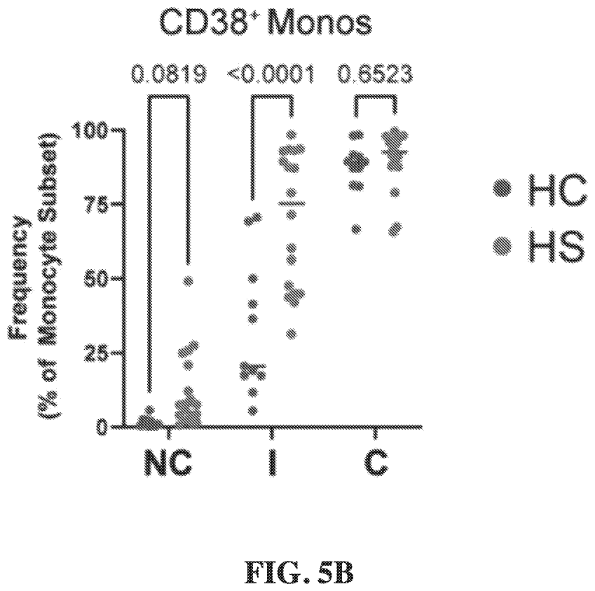

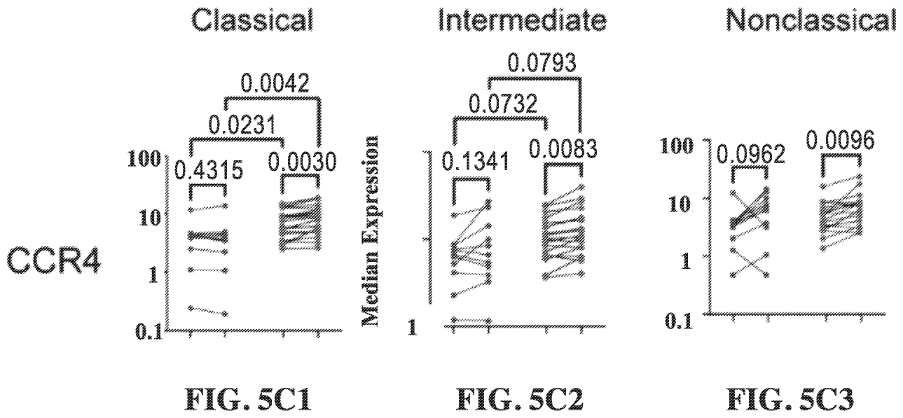

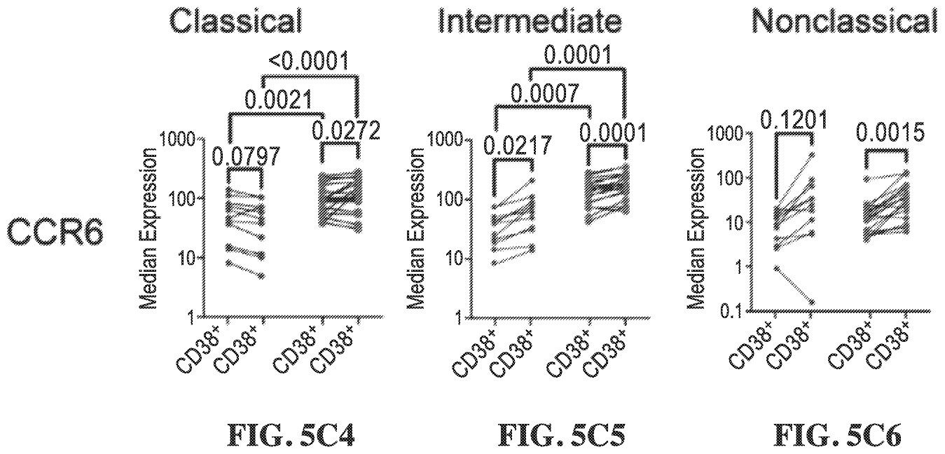

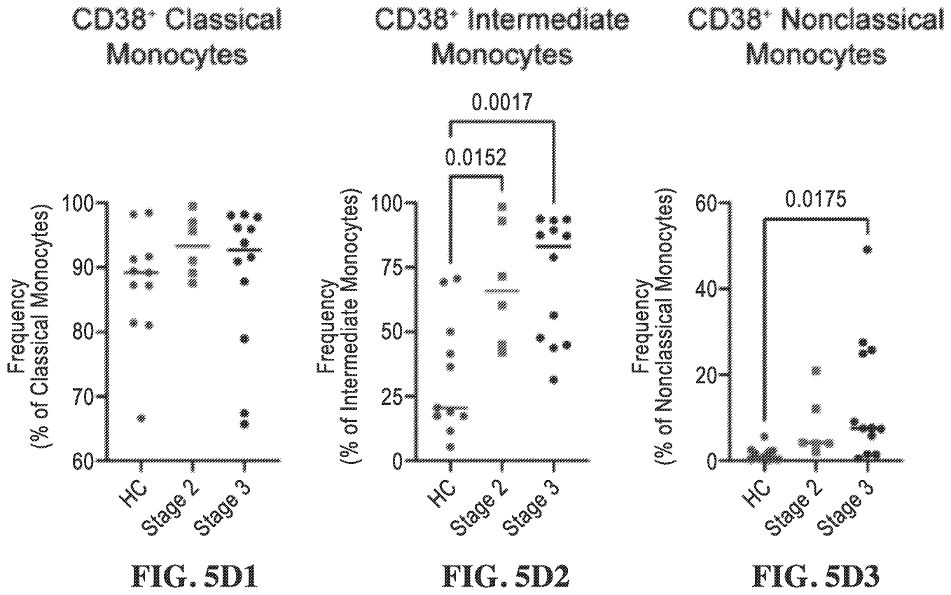

The background level of CD38 expression in monocyte subsets was defined using the gating strategy shown in FIG. 5A. Classical monocytes from HC patients were almost entirely CD38+(FIG. 5A), while the frequency of CD38+ intermediate monocytes was elevated in HS patients compared to HC (FIG. 5B). A discrete population of CD38+ non-classical monocytes was present in only some patients with HS (p=0.0819). CCR4 and CCR6 expression in HS classical monocytes showed a significant disease association increase regardless of CD38 expression (FIGS. 5C1 and 5C4). Only CCR6 expression showed significant disease association increase in HS intermediate monocytes, whereas CCR4 expression approached significant regardless of CD38 status (FIGS. 5C2 and 5C5). CCR4 and CCR6 expression levels increased in HS CD38+ monocytes compared to CD38-monocytes, whereas only CCR6 expression increased in HC CD38+ intermediate monocytes. No changes were found in CCR4 and CCR6 expression between HC and HS nonclassical monocytes (FIGS. 5C3 and 5C6). Thus, HS CD38+ monocytes show increased skin-homing capacity, and intermediate monocytes, and not classical or non-classical monocytes, showed elevated CD38+ in early Hurley stage II (FIG. 5D1-3) Altogether, the data highlight enhanced expression of skin homing CCRs and CD38+ on multiple circulating immune cell subsets in HS patients, which may serve as early biomarkers for HS. 1.2 Characterization of CD38 expression in lesional HS skin

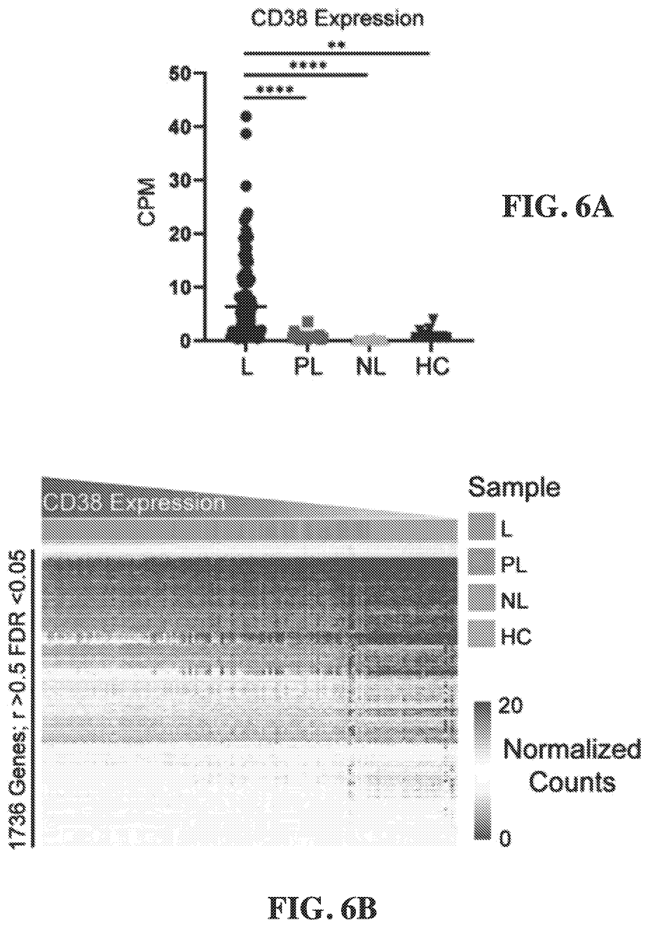

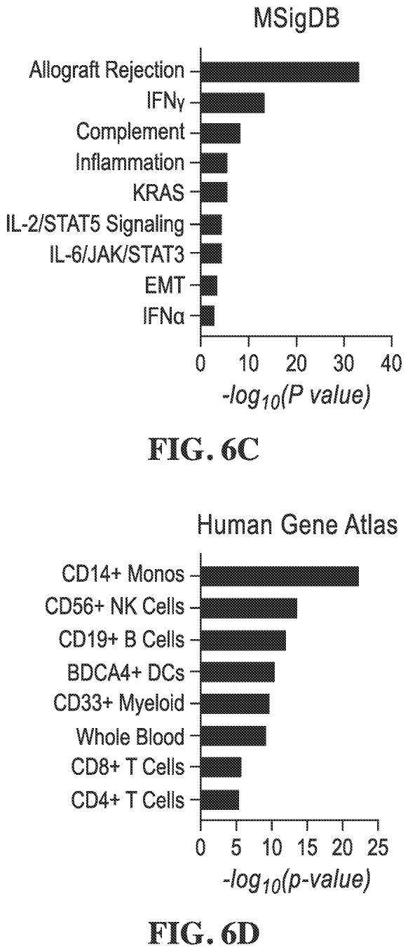

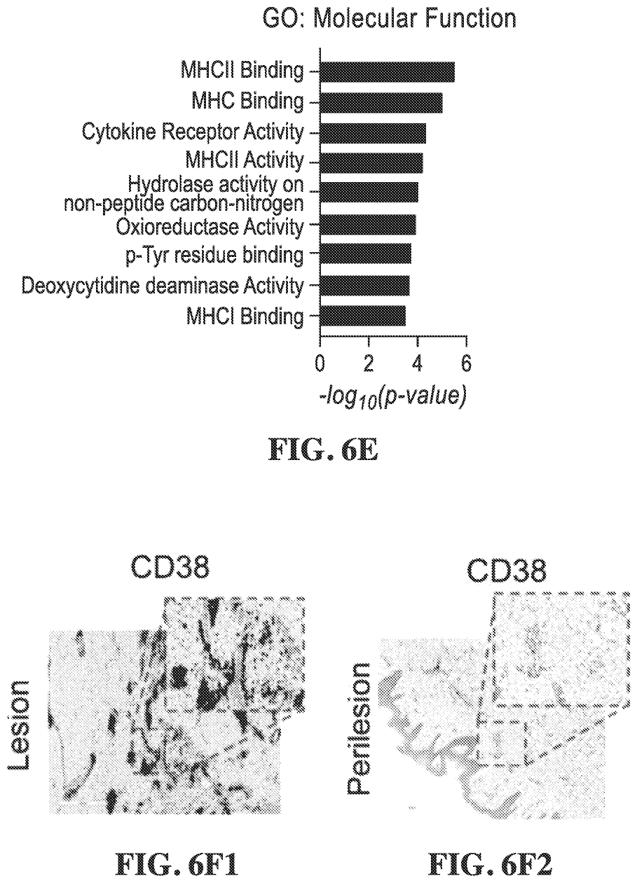

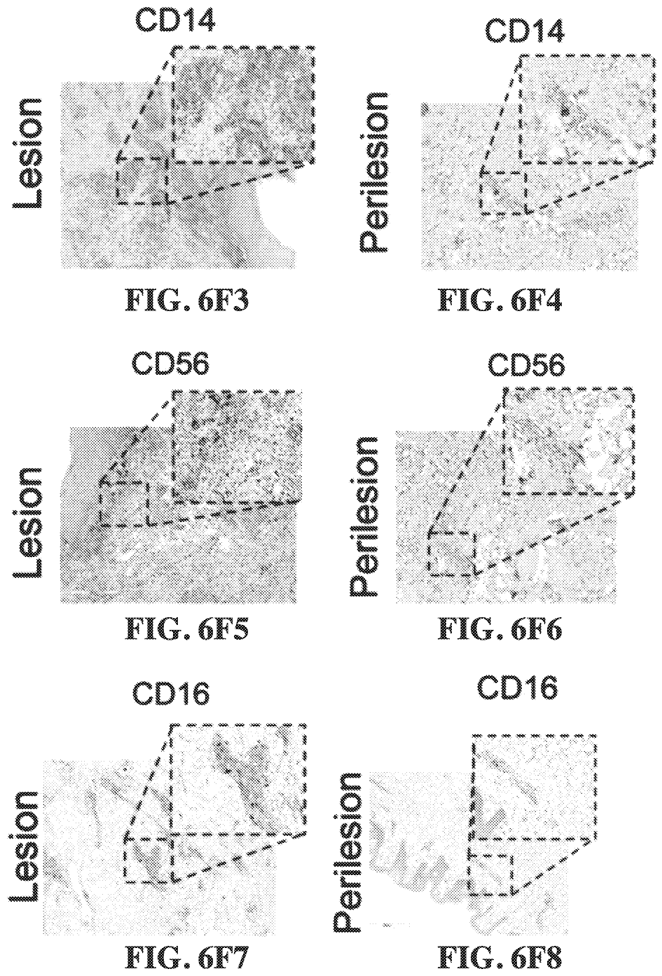

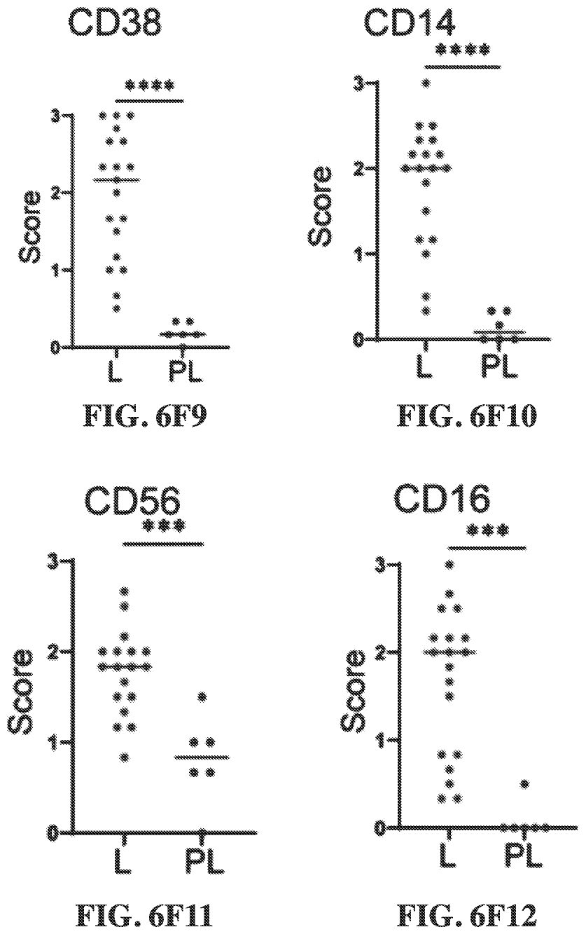

An HS-Omics database with aggregated publicly available bulk and single-cell RNA sequencing data from HS and HC tissue samples were analyzed to determine CD38 expression in HS lesional (L) versus perilesional (PL) tissue and nonlesional (NL), as well as tissue from HC skin (FIG. 6A). A total of 1736 genes showed significant correlation with CD38 expression in heatmap gene expression data (r >0.5; FDR <0.05) (FIG. 6B). Gene identification was enriched by were identified using MSigDb (FIG. 6C), the Human Gene Atlas (FIG. 6D), and gene ontology:molecular function (GO:MF) (FIG. 6E) higher in lesional HS tissue compared to perilesional tissue. The genes enriched by MSigDB pathways are associated with allograft rejection, IFNγ signaling, complement activity, IL2/STAT5, and IL6 signaling (FIG. 6C). An analysis using the human gene atlas found that CD38-correlated genes were enriched for those expressed by CD14+ classical monocytes, NK cells, and DCs (FIG. 6D). These cell types were reduced in blood from HS patients compared to HCs (FIG. 2A1 2D1-3, 2E2), suggesting immune infiltration to HS skin lesions.

### 1.3 Immunohistochemical Analysis of CD38, Monocyte (CD14 and CD16) and NK Cell (CD16 and CD56) Markers in HS and HC Tissue Samples

Immunohistochemistry analysis was performed using CD38 and monocyte (CD14 & CD16) and NK cell (CD16 & CD56) markers on 19 lesional and 6 perilesional HS tissue samples (FIG. 6F1-12). CD38 and monocyte and NK markers were dramatically higher in the lesional-skin compared to perilesional-skin (FIG. 6F9-12). The staining pattern of CD38 suggested that immune and stromal cells express CD38 in HS lesional-skin. These results also provide compelling evidence that the observed decrease in circulating classical monocytes, NK cells, and DCs is associated with immune infiltration into HS lesional-skin.

### 1.4 Imaging Mass Cytometry (IMC)

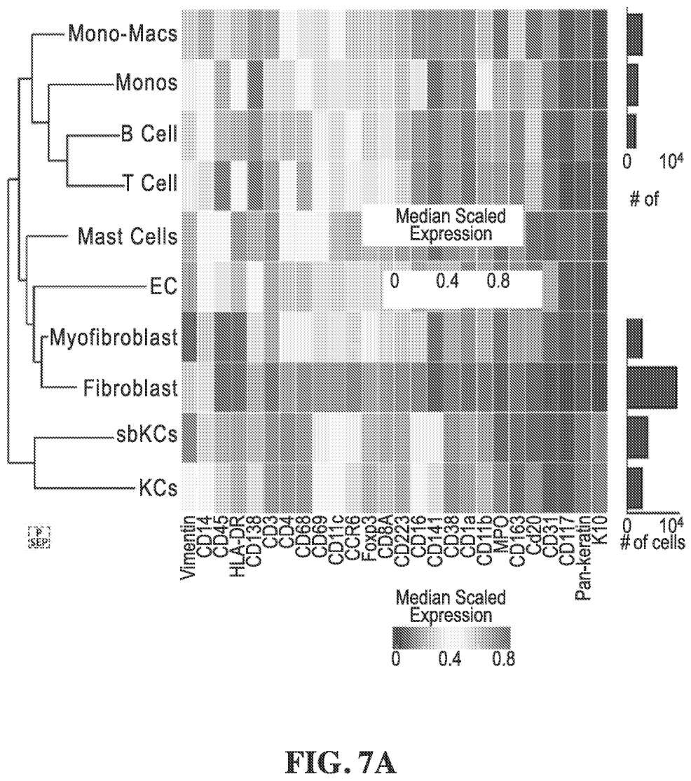

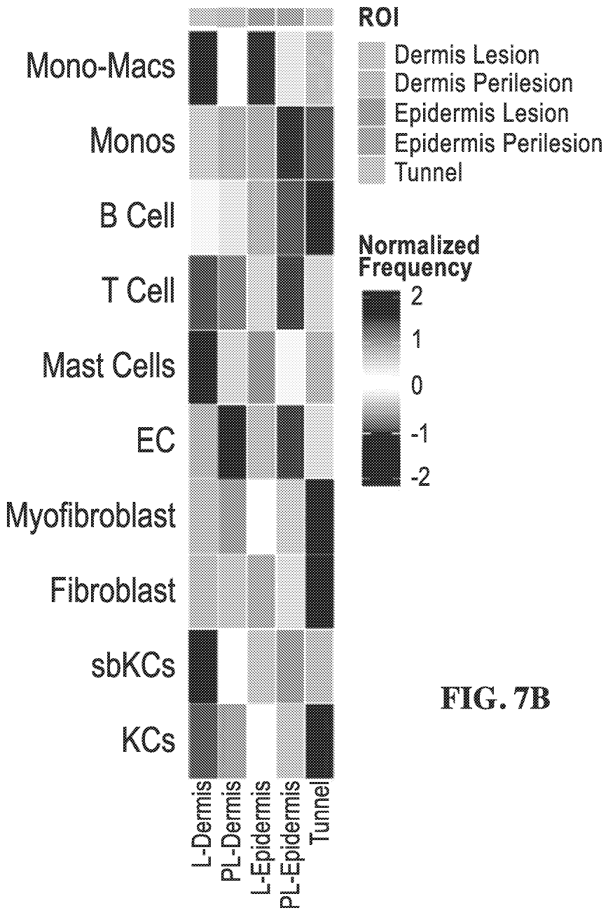

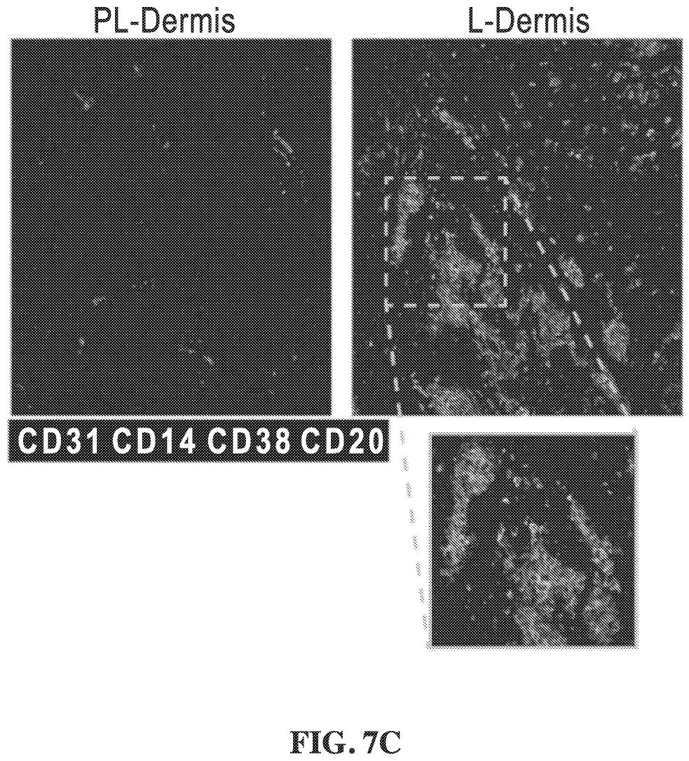

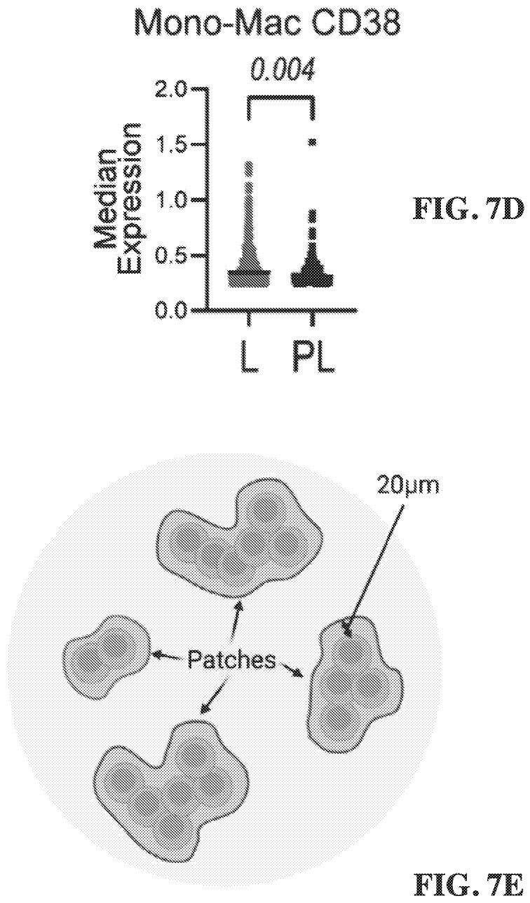

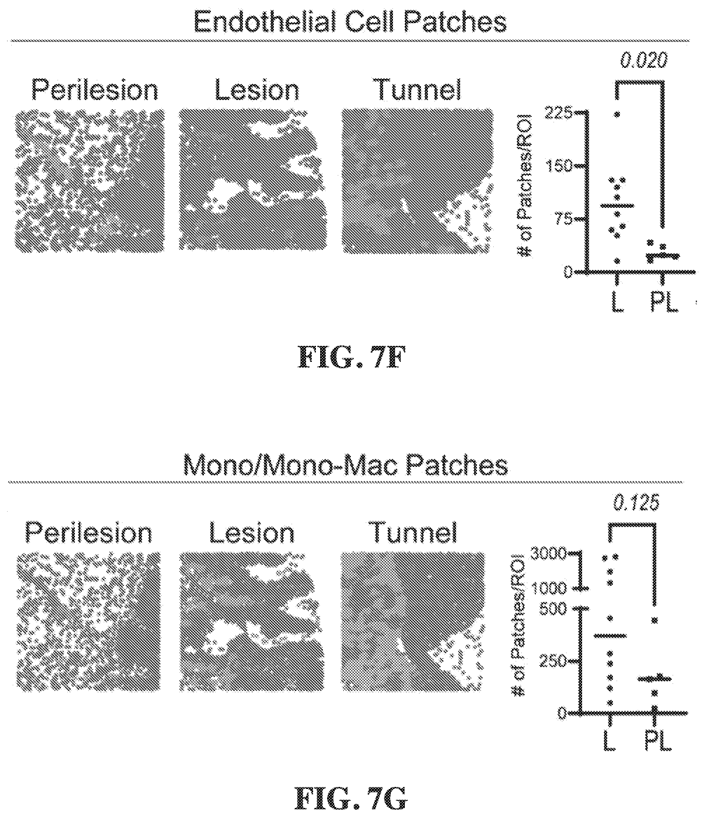

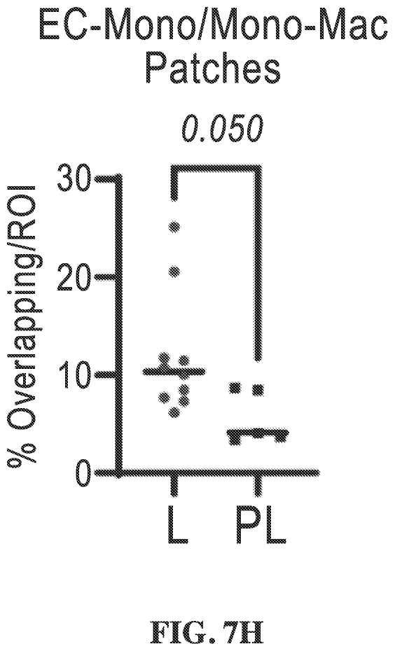

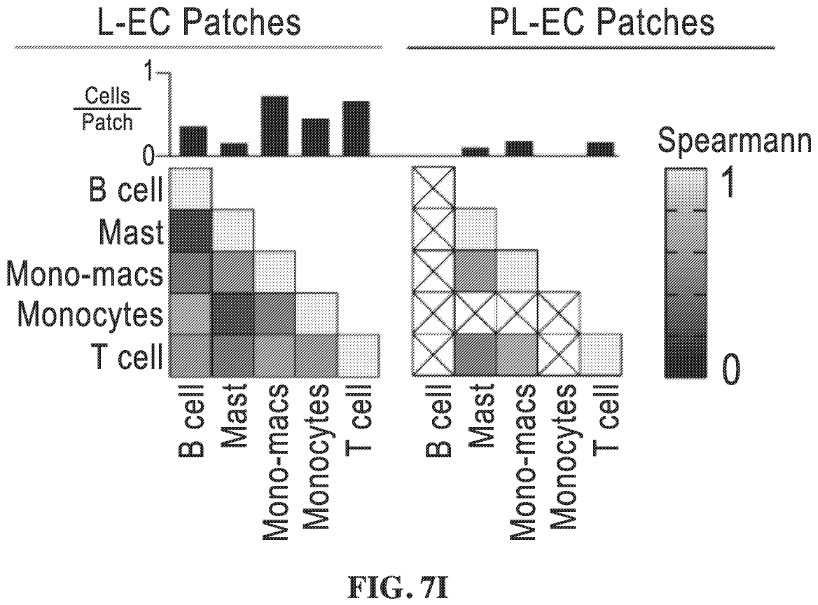

A panel of 31 antibodies was used with imaging mass cytometry to identify cell types from 16 regions on interest in 3 lesional and 3 perilesional HS tissue samples (FIG. 7A). Only one cell cluster expressed both CD14 and CD16 along with requisite monocyte markers (CD11b+ CD68− HLA-DR+ CD11c−), which indicated that monocytes clusters in HS skin are predominantly classical monocytes.

Comparing the frequencies of immune cells in different regions of interest showed that monocytes were enriched in lesional dermis and -tunnel regions; monocyte-derived macrophages were enriched in lesional-epidermis regions, T cells were enriched in lesional-dermis and -tunnel regions; B Cells were most enriched near lesional-tunnels; and endothelial cells were enriched in lesional-dermis, -epidermis, and -tunnel regions (FIG. 7B).

Comparing CD38 staining between lesional and perilesional HS tissue, showed a dearth of CD38 expression in perilesional regions of interest and an abundance in lesional regions of interest. CD38 exhibited co-staining with CD31 and CD14 in lesions-dermal regions of interest (FIG. 7C). In addition, some B cells in these regions also expressed CD38, which did not co-stain with other markers in our panel (FIG. 7C). CD38 expression was quantified in monocytes and monocyte-derived macrophages in HS lesional regions (dermis, epidermis, and tunnel) and perilesional regions (dermis and epidermis). Using a median expression cutoff of 0.25 to define positive staining, the expression of CD38 in monocyte-derived macrophages was significantly higher in lesions than in perilesional tissue samples (FIG. 7D). Notably, only 11 CD38+ monocytes were found in perilesional tissue samples. Taken into consideration with our CyTOF data, these findings suggest that CD38+ classical monocytes from the blood are preferentially trafficking to HS skin lesions over perilesional-skin. Together, these data show that CD38 is expressed by monocytes, monocyte-derived macrophages, and endothelial cells more highly in HS lesional-skin than in perilesional-skin.

Using a patch detection algorithm and endothelial cells, combined monocytes and monocyte-derived macrophages as the reference clusters. The patches are defined as the space encompassed by a concave hull drawn around a reference cluster that extends 20 μm from the edge of a reference cell (FIG. 7E). The number of endothelial cell patches was significantly higher in HS lesional skin compared to perilesional skin (FIG. 7F). There was also a trend of increasing combined monocytes and monocyte-derived macrophages patches is lesional tissue relative to perilesional tissue (p=0.125) (FIG. 7G).

Comparing the proportion of endothelial cell and combined monocytes and monocyte-derived macrophages patches that spatially overlapped, showed significantly more overlap in HS lesional-skin than in perilesional-skin, suggesting that endothelial cells and monocytes and monocyte-derived macrophages share a microenvironment in HS lesional-skin (FIG. 7H).

Characterization of the immune cell types in lesional and perilesional endothelial cell microenvironments identified a higher frequency and diversity of immune cells in lesional endothelial cell microenvironments, the one exception being mast cells, which were present equally (FIG. 7I). Perilesional endothelial cell microenvironments lacked B cells and monocytes, and the correlation of immune cell frequencies was similar between the cells that were present in both lesional and perilesional endothelial cell microenvironments (FIG. 7I).

Comparison of the median expression of cytokines, signaling molecules, and functional markers in the immune cells present in lesional and perilesional microenvironments identified monocyte-derived macrophages in endothelial cell patches with higher levels of CCR6 expression (Box1) and lower levels of TGFβ expression (Box 2) than perilesional monocyte-derived macrophages (FIG. 7J). Lesional endothelial cells also expressed lower levels of TGFβ (Box 3) (FIG. 7J). Even mast cells, which had similar frequencies in lesional and perilesional endothelial cell microenvironments, had differing expression of functional markers (FIG. 7J). Taken together, these data show that the number of endothelial cell patches, immune cell composition, and functional status of immune cells differ between HS lesional and perilesional endothelial cell microenvironments.

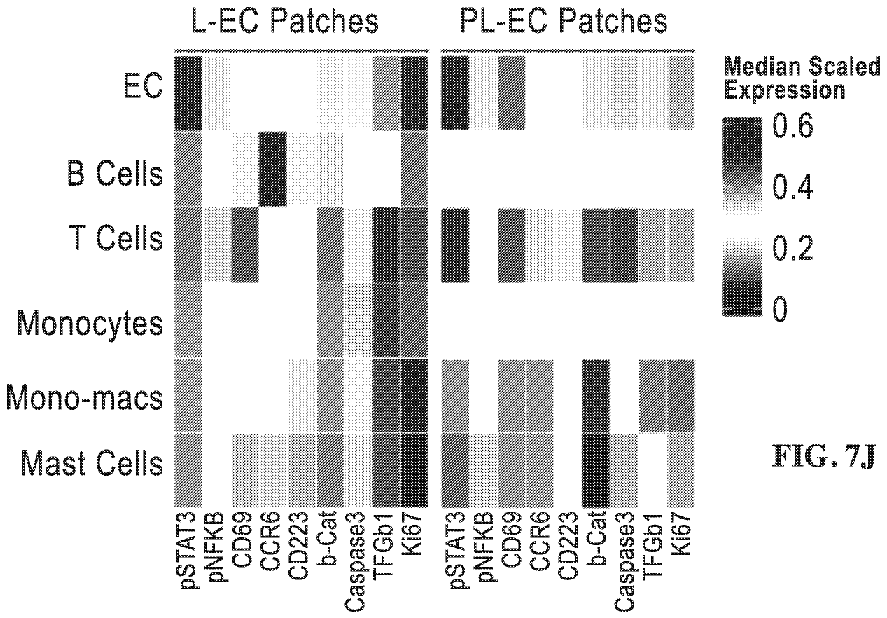

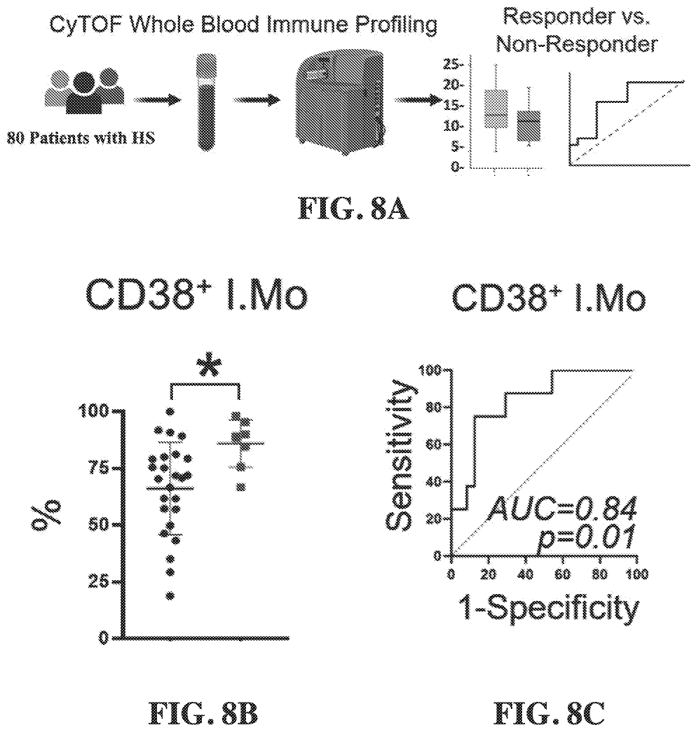

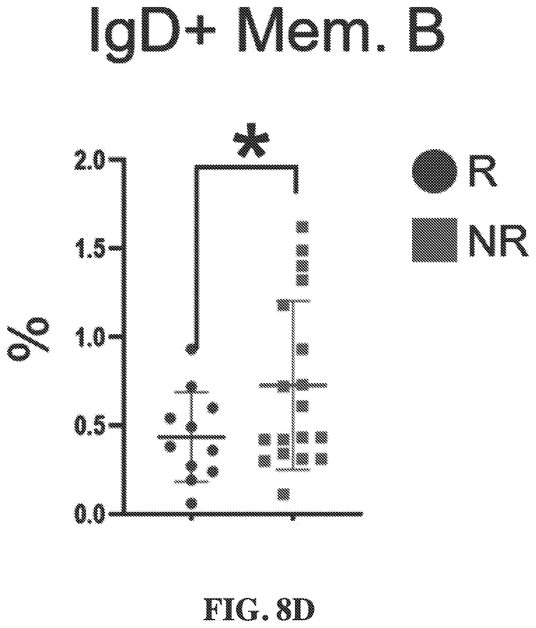

Biomarkers are needed that can predict whether HS patients will respond to anti-TNFα therapy. CyTOF immune profiling was performed to identify immunome predictors of anti-TNFα therapy response (FIG. 8A). HS patients that failed to respond to anti-TNFα therapy had higher frequency of CD38+ intermediate monocytes (I.mos) out of the total intermediate monocyte cell population (FIG. 8B). A receiver operating characteristic curve (ROC) analysis demonstrated that CD38+ intermediate monocytes frequency predicts TNFα therapy response (FIG. 8C). The frequency of IgD+ memory B cells (Mem. B) was also predictive of TNFα therapy response (FIG. 8D). The immune profiling data suggests that a high frequency of CD38+ intermediate monocytes and memory B cells is predictive of TNFα non-responders.

Collectively, the experimental data in Example 1 supports that anti-CD38 targeting with a monoclonal antibody may exert a therapeutic effect by acting on monocytes, B cells, NK cells, and/or endothelial cells.

Although the disclosure of the invention has been described in detail for purposes of clarity and understanding, it will be obvious to those with skill in the art that certain modifications can be practiced within the scope of the appended claims. All publications and patent documents cited herein are hereby incorporated by reference in their entirety for all purposes to the same extent as if each were so individually denoted.

Throughout this specification, unless the context requires otherwise, the word “comprise,” or variations such as “comprises” or “comprising,” will be understood to imply the inclusion of a stated step or element or integer or group of steps or elements or integers but not the exclusion of any other step or element or integer or group of elements or integers.

1. A method for the treatment or prevention of Hidradenitis suppurativa (HS), the method comprising administering a therapeutically effective dose of an anti-CD38 antagonist to a subject with HS or likely to develop HS to treat or ameliorate one or more symptoms of HS.

2. The method of claim 1, wherein the anti-CD38 antagonist is an CD38 antisense molecule that inhibits transcription or translation of CD38, an anti-CD38 antibody, or antigen-binding fragment thereof, an antiCD38 antibody conjugated to a cytotoxic agent, or combinations thereof, that specifically binds to CD38, or a small molecule that inhibits the activity of CD38.

3. The method of claim 2, wherein the anti-CD38 antagonist is an anti-CD38 antibody, or antigen-binding fragment thereof that specifically binds to CD38 and inhibits the activity of CD38.

4. The method of claim 2, wherein the anti-CD38 antagonist specifically antagonizes, destroys or depletes cells bearing CD38.

5. The method of claim 4, wherein the anti-CD38 antagonist is an anti-CD38 antibody, or antigen-binding fragment thereof that specifically binds to CD38 on a CD38 positive cell.

6. The method of claim 4, wherein the treatment comprises reducing the activity or cell numbers of CD38 positive cells selected from: circulating NK cells, memory B cells, monocytes, skin and lesional monocytes and monocyte-derived macrophages, and skin and lesional skin endothelial cells.

7. The method according to claim 6, wherein the treatment comprises reducing the activity or cell numbers of CD38 positive cells selected from: skin-tropic intermediate monocytes, skin and lesional monocyte-derived macrophages, lesional skin endothelial cells and combinations thereof.

8. The method of claim 1, wherein the anti-CD38 antagonist is an anti-CD38 antibody, or antigen binding fragment thereof.

9. The method of claim 8, wherein the anti-CD38 antibody, or antigen binding fragment thereof is selected from the group consisting of: Ab2, Ab3, Ab4, Daratumumab®, MOR202, HexaBody®-CD38 (Genmab/Janssen), SARCLISA® (Sanofi; isatuximab-irfc), anti-CD38 SIFbody, TSK011010 (CASI), or an antigen-binding fragment thereof of any of the foregoing anti-CD38 antibodies.

10. The method of claim 8, wherein the anti-CD38 antibody is Daratumumab® or an antigen-binding fragment thereof, or SARCLISA® (Sanofi; isatuximab-irfc) or an antigen-binding fragment thereof.

11. The method of claim 8, wherein the anti-CD38 antagonist is Daratumumab® or SARCLISA® (Sanofi; isatuximab-irfc), or an antigen-binding fragment thereof, each of the foregoing conjugated to a cytotoxic agent.

12. The method of claim 1, wherein treatment of the HS patient with a therapeutically effective anti-CD38 antagonist reduces the subject's HisSCR by at least a 50% in the total inflammatory lesion (abscess and inflammatory nodule) count (AN count).

13. Use of a composition comprising an anti-CD38 antagonist for the treatment of Hidradenitis suppurativa (HS) in a subject in need thereof.

14. The use according to claim 13, wherein the anti-CD38 antagonist comprises an anti-CD38 antibody, or an antigen binding fragment thereof, or an anti-CD38 antibody conjugated to a cytotoxic agent.

15. The use according to claim 14, wherein the anti-CD38 antibody, or an antigen binding fragment thereof is a human or humanized anti-CD38 antibody, or an antigen binding fragment thereof.

16. The use according to claim 15, wherein the anti-CD38 antibody is Daratumumab® or an antigen-binding fragment thereof, or SARCLISA® (Sanofi; isatuximab-irfc) or an antigen-binding fragment thereof.

17. The use according to claim 16, wherein the anti-CD38 antibody is SARCLISA® (Sanofi; isatuximab-irfc) or an antigen-binding fragment thereof.

18. The use according to claim 16, wherein the anti-CD38 antibody is Daratumumab® or an antigen-binding fragment thereof.

19. A kit for the treatment or prevention of Hidradenitis suppurativa (HS), comprising an anti-CD38 antagonist that specifically binds to human CD38, or inhibits its expression or activity, optionally combined with a secondary active agent, as well as instructions for the use of the anti-CD38 antagonist or optionally combination of active agents.

20. The kit of claim 19, wherein the anti-CD38 antagonist is a human or humanized anti-CD38 antibody or antigen-binding fragment thereof.

21. The kit of claim 19, wherein the optional secondary active agent is an antibiotic, an anti-inflammatory, a chemotherapeutic agent, an immunosuppressant, or combinations thereof.
